(19) United States
(12) Patent Application Publication
(10) Pub. No.: US 2022/0273556 A1
(43) Pub. Date: Sep. 1, 2022

(54) On Demand Drug Release System

(71) Applicant: Northeastern University, Boston, MA (US)

(72) Inventors: Thomas WEBSTER, Barrington, RI (US); Siddhant SHARMA, Floral Park, NY (US)

(21) Appl. No.: 17/680,152

(22) Filed: Feb. 24, 2022

Related U.S. Application Data
(60) Provisional application No. 63/153,322, filed on Feb. 24, 2021.

(51) Int. Cl. A61K 9/00; A61K 47/02; A61K 47/34; A61L 27/54; A61K 47/42

(52) U.S. Cl. CPC A61K 9/0024; A61K 47/02; A61K 47/34; A61L 27/54; A61K 47/42; B33Y 70/00

(57) ABSTRACT

An on-demand drug release system provides an implantable drug delivery platform including a drug embedded in a controllable drug release structure. The drug release structure binds or compartmentalizes the drug until release of the drug is initiated by absorption of a near infrared release signal provided by a health care professional. The system allows more effective therapeutic use of opioids and other drugs, and prevents their abuse.

### CROSS-REFERENCE TO RELATED APPLICATIONS

This application claims priority to U.S. Provisional Application No. 63/153,322, filed 24 Feb. 2021, which is incorporated by reference herein in its entirety.

### BACKGROUND

A global opioid epidemic poses a crisis in many countries. Opioids are substances derived from the opium poppy or synthetic analogues thereof. Some examples are fentanyl, morphine, heroin, tramadol, oxycodone, vicodin, and methadone. Opioids have the potential to cause substance dependence characterized by strong physical and psychological addiction, which can have harmful or devastating life consequences. Abuse of opioids can cause increased tolerance and physical withdrawal symptoms when the use of opioids is discontinued. Dependence on prescription opioids can develop following the treatment of chronic pain, such as after surgery or treatment for an injury or a disease. Opioids are responsible for a high proportion of fatal drug overdoses around the world.

New forms of drug delivery that provide controllable drug release are needed to provide better management of opioids and other therapeutics.

### SUMMARY

The present technology provides a drug delivery platform with a therapeutic agent securely embedded inside the platform. The therapeutic agent is held inside the platform until a controlled release is initiated. The drug delivery platform is implantable in a subject and configured to receive a release signal. When the release signal is received, at least a portion of the therapeutic agent is released in the subject, providing an on-demand drug release system.

The release signal can be provided by a health care provider. The platform and release signal can be configured to provide the health care provider control over release of the therapeutic agent and to prevent any unplanned release of the therapeutic agent. In another example, the release signal can be delivered by a controller linked to equipment implanted in the subject's body or worn by the subject, with the equipment capable of measuring one or more parameters.

The drug delivery platform includes an outer 3D structure. The outer 3D structure can be fabricated by 3D printing, by other manufacturing techniques, or by a combination of techniques. The outer 3D structure includes a shape memory polymer (SMP) with a receiving agent capable of absorbing a bandwidth of electromagnetic radiation (EM) and transducing the EM to the SMP, thereby causing a shape change in at least a portion of the 3D structure.

The receiving agent is a thermal transducing agent capable of absorbing near infrared (NIR) radiation and producing thermal motion of its molecular structure or of structures of nearby molecules. The thermally absorbing agent can be referred to as a photothermally absorbing agent. A NIR wand or a NIR emitter, preferably accessible or usable only by a trained health care provider, can provide control over therapeutic release by the system. The system can prevent abuse of analgesics by allowing control of release only to the healthcare provider, such that release of pain medication is managed more effectively. While other bandwidths of EM can be utilized, NIR radiation provides an example of EM that can safely penetrate into the subject, and NIR can be easily applied external to the subject by the health care provider.

The outer 3D structure can include a matrix between the structure and the therapeutic agent. The matrix can include or consist of a biodegradable polymer. The biodegradable polymer can encapsulate the therapeutic agent or can be associated with the therapeutic agent. For example, the biodegradable polymer can be a self-assembled biodegradable polymer which may include amphiphilic monomers.

The present technology can be further summarized by the following list of features.

1. An implantable drug release structure comprising:

a shape memory polymer having a glass transition temperature greater than about 37° C.;

a thermal transduction agent capable of absorbing near infrared light and inducing thermal motion in response thereto, wherein the thermal transduction agent is non-covalently associated with said shape memory polymer; and

a drug non-covalently associated with the implantable drug release structure below said glass transition temperature.

2. The implantable drug release structure of claim 1, wherein the drug is disposed within a polymer matrix comprising the shape memory polymer.
3. The implantable drug release structure of claim 2, wherein the drug is embedded within the polymer matrix.
4. The implantable drug release structure of any of the preceding claims, wherein the drug is disposed within a compartment formed by the drug release structure in a closed configuration, and wherein said compartment opens above the glass transition temperature to release the drug.
5. The implantable drug release structure of any of the preceding claims, wherein the drug is associated with a biodegradable polymer, said biodegradable polymer associated with the implantable drug release structure.
6. The implantable drug release structure of claim 5, wherein the biodegradable polymer comprises an amphiphilic peptide.
7. The implantable drug release structure of any of the preceding claims, wherein the drug is associated with nanoparticles, said nanoparticles associated with the implantable drug release structure.
8. The implantable drug release structure of any of the preceding claims, wherein the thermal transduction agent comprises graphene.
9. The implantable drug release structure of any of the preceding claims, wherein the thermal transduction agent is present in an amount up to about 20 weight % of the implantable drug release structure.
10. The implantable drug release structure of any of the preceding claims, wherein the shape memory polymer comprises an epoxy monomer, an aliphatic diamine crosslinker, and a crosslinking modulator.
11. The implantable drug release structure of claim 10, wherein the crosslinking modulator comprises decylamine.
12. The implantable drug release structure of any of the preceding claims, wherein the glass transition temperature is about 45° C.
13. The implantable drug release structure of any of the preceding claims, wherein the drug is an analgesic agent, an anti-inflammatory agent, an antibiotic, or a combination thereof.
14. The implantable drug release structure of any of the preceding claims, wherein the shape memory polymer changes form when heated to a temperature above the glass transition temperature, said change in form leading to release of the drug from the implantable drug release structure.
15. The implantable drug release structure of any of the preceding claims, wherein the thermal transduction agent is associated with the shape memory polymer via π-π interactions, cation-π interactions, ionic interactions, van der Waals interactions, hydrogen bonding interactions, or a combination thereof.
16. A method of administering a drug to a subject, the method comprising:

(a) providing an implantable drug release structure comprising:
a shape memory polymer having a glass transition temperature greater than about 37° C.;
a thermal transduction agent capable of absorbing near infrared light and inducing thermal motion in response thereto, wherein the thermal transduction agent is non-covalently associated with said shape memory polymer; and
a drug non-covalently associated with the implantable drug release structure below said glass transition temperature;

(b) implanting the implantable structure in the subject; and

(c) irradiating the subject with near infrared radiation in a body region comprising the implanted drug release structure, whereby the thermal transduction agent absorbs the near infrared radiation. thereby inducing a shape change of the implanted drug release structure and releasing the drug.

17. The method of claim 16, wherein step (c) is performed by trained medical personnel or in a doctor's office, hospital, or other medical facility.
18. The method of claim 16 or 17, further comprising, prior to step (b):

(b0) performing a surgical procedure at a site in the subject's body; and wherein step (b) comprises implanting the implantable drug release structure at or near the surgical site.

19. The method of any of claims 16-18, wherein the implantable drug release structure is an implantable medical device or forms a portion of an implantable medical device.
20. The method of claim 19, wherein the implantable medical device is an orthopedic device and the surgical procedure is an orthopedic surgical procedure.
21. The method of any of claims 16-18, wherein the drug is an analgesic agent, an anti-inflammatory agent, an antibiotic, or any combination thereof.
22. A method of making an implantable drug release structure, the method comprising the steps of:

(a) providing an ink for 3D printing, the ink comprising a shape memory polymer, a thermal transduction agent, and a drug;

(b) printing the ink into a 3D object having a first shape;

(c) heating the object to above a glass transition temperature of the shape memory polymer;

(d) changing the heated object's shape from the first shape to a second shape; and

(e) cooling the object to below the glass transition temperature while maintaining the second shape.

23. A kit for preparing the implantable drug release structure of any of claims 1-15, the kit comprising:
(i) an ink for 3D printing, the ink comprising a shape memory polymer, a thermal transduction agent; and
(ii) instructions for preparing an implantable drug release structure using the ink and the method of claim 22; and
(iii) optionally one or more drugs for addition to the ink and use in the method to prepare the implantable drug release structure; and
(iv) optionally one or more implantable medical devices for use as a substrate during said 3D printing.
   24. An implantable medical device comprising one or more implantable drug release structures of any of claims 1-15 attached to the device or present as a coating or portion thereof of the device.
   25. The implantable medical device of claim 24, wherein the device comprises a remotely activatable near IR light source that, when activated, causes release of the drug present in the one or more implantable drug release structures.

As used herein, the terms infrared (IR) radiation, IR electromagnetic radiation, and IR light refer to electromagnetic radiation having wavelengths in the range from about 700 nm to about 1 mm. Visible electromagnetic radiation (visible light) and optical electromagnetic radiation refer to electromagnetic radiation having wavelengths in the range from about 380 nm to about 700 nm. NIR refers to electromagnetic radiation having wavelengths in the range from about 700 nm to about 1.4 μm. Short-wavelength IR refers to electromagnetic radiation having wavelengths in the range from about 1.4 μm to about 3 μm. Mid-IR refers to electromagnetic radiation having wavelengths in the range from about 3 μm to about 8 μm. Long-wavelength IR refers to electromagnetic radiation having wavelengths in the range from about 8 μm to about 15 μm. Thermal-IR refers to electromagnetic radiation having wavelengths in the range from about 3 μm to about 30 μm. Far-IR (FIR) refers to electromagnetic radiation having wavelengths in the range from about 15 μm to about 1 millimeter.

As used herein, the term “electromagnetic radiation” or “EM” can be used interchangeably with the term “light”. Light discussed herein can be unpolarized or polarized in a linear, circular, or elliptical polarization. The term “elliptical polarization” is referred to herein as “circular polarization”.

The technology can provide for a subject to control a release of the therapeutic agent in the subject's body. In a subject-controlled mode, the release signal can be provided by the subject. For example, the implanted technology with a subject's control of the release signal can take the place of a patient-controlled analgesia pump that typically provides a subject control of intravenous (IV) pain medicine when a subject needs it. In another example, electrical signals from the subject's body can provide a release signal.

As used herein, the term “nanostructure” or “nanomaterial” refers to a structure having at least one dimension on the nanoscale, i.e., from about 1 nm to about 999 nm. Nanostructures can include, but are not limited to, nanosheets, nanotubes, nanoparticles, nanospheres, nanocylinders, nanowires, nanocubes, nanowalls, nanoshapes, and combinations thereof. As used herein, a 2D structure, a 2D layer, and a 2D material means a composition including a thickness (Z) on the nanoscale from about atomic thickness (about 0.3 nm to about 1.5 nm thick) up to about 10 nm. The 2D layer/structure/material can extend in the X/Y distances for any distance (e.g., beyond millimeters).

As used herein, the term “microstructure” or “micromaterial” refers to a structure having at least one dimension on the microscale, that is, at least about 1 micrometer to about 999 micrometers.

As used herein, the term “room temperature” refers to a temperature in the range from about 20° C. to about 25° C. As used herein, the term “IUPAC room temperature” refers to a temperature at about 25° C. As used herein, normal human body temperature refers to a core body temperature in the range from about 36.0° C. to about 37.0° C.

As used herein, the term “4D transformation” refers a shape change over time by a 3D structure either caused by a release signal provided by a health care provider or provided without a medical intervention. As used herein, 4D printing is an additive manufacturing (3D printing) process used to fabricate a pre-designed, self-assembling structure with the ability to transform over time to a different structure in response to a signal such as NIR or other EM supplied to the structure, such as a signal applied to a part of the body after implantation.

As used herein, the term “amphiphilic” refers to a compound including an organic cation or anion which includes a long unbranched hydrocarbon chain, (e.g., CH₃(CH₂)_{f}CO₂⁻, CH₃(CH₂)_{f}N⁺(CH₃)₃ (f>7), CH₃(CH₂)_{f}SO₃⁻. The presence of distinct polar (hydrophilic) and nonpolar (hydrophobic) regions in the molecule can promote the formation of micelles in dilute aqueous solution. As used herein, the term “amphiphilic molecule” and “amphiphilic monomer” refers to a molecule or monomer, respectfully including a polar water-soluble functional group attached to a water-insoluble function group (e.g., hydrocarbon chain). An amphiphilic molecule can be a surfactant.

As used herein, the term “alkyl” refers to univalent groups derived from alkanes by removal of a hydrogen atom from any carbon atom —CₑH₂₊₁. The groups derived by removal of a hydrogen atom from a terminal carbon atom of unbranched alkanes form a subclass of normal alkyl (n-alkyl) groups H(CH₂)ₑ. The groups RCH₂, R₂CH (R≠H), and R₃C (R≠H) are primary, secondary and tertiary alkyl groups, respectively, and an “alkyl” can be branched or unbranched. The term “alkyl” includes substituents on the alkane.

As used herein, the term “alkenyl” refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may be bonded on one or more carbons that are included or not included in one or more double bonds.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1A shows the chemical structure of an example shape memory polymer (SMP) and an example thermally absorbing agent of photothermally responsive graphene. Dotted lines below the graphene depict π-π interactions between the SMP and the graphene. FIG. 1B shows dynamic viscosity of 3D nanocomposite inks with different graphene (weight %) contents measured by rheometer at room temperature. FIG. 1C shows Raman spectra of nanocomposites with different graphene contents, and the Raman spectrum of graphene is shown at bottom. FIG. 1D shows DSC curves of nanocomposites with different graphene contents and pure SMP (0% graphene, bottom).

FIG. 2A shows a plot of shape changing time versus triggering temperature. FIG. 2B shows load-extension curves of nanocomposite constructs with 16% graphene and pure SMP (0%) characterized at room temperature via uniaxial tensile testing. FIG. 2C shows tensile modulus of different nanocomposite constructs including 0% graphene (weight %), 4%, 8%, 12%, 16%, and 20%. FIGS. 2D-2I show cross-sectional SEM images of different samples. The scale bar at lower right of each figure is 200 μm. The inset in each image shows an enlarged morphology with a 10 μm scale bar. FIG. 2D shows 0% graphene. FIG. 2E shows 4% graphene. FIG. 2F shows 8% graphene. FIG. 2G shows 12% graphene. FIG. 2H shows 16% graphene. FIG. 2I shows 20% graphene (weight %).

FIGS. 3A-3B show example schematics of NIR sensitive 4D transformation. In FIG. 3A, the printed nano-composite construct has an initial shape (I, far left). After exposure to NIR illumination (T>Tg, Thermal/photothermal), the construct is changed to a temporary shape by an external force, and the construct's shape is fixed (III) at room temperature (Phase 1, top). Once heating up to the Tg through NIR exposure (locally photothermal), the temporary shape of the construct can gradually return to its original shape (IV-V-I). During this photothermal process, the NIR illumination can control the shape-changing position and transformation time (Phase 2, right and Phase 3, bottom). Multiple position illumination can return phase 3 to the original shape at far left. In FIG. 3B, an original shape (top) is formed into a temporary shape (bottom-left) by an external force, which can be gradually returned (i.e., part-by-part, bottom-right) to original shape via remote/dynamic control, such as through application of NIR laser. Multiple position illumination can return the shape at bottom-right to the original shape at top.

FIG. 4A shows images of dynamically controllable transformation of 4D printed constructs including NIR sensitive 4D transformation behavior of nanocomposite models, including a blooming flower, hand gesture, exerciser, controllable circuit switch, folded brain, and dilated heart. The shape of each model is shown as it is dynamically and precisely controlled under NIR exposure left to right. FIG. 4B shows an example of an on-demand drug release system with a 3D structure holding a therapeutic agent (left) before a release of the therapeutic agent via application of NIR and after the release (right) of the therapeutic agent. FIG. 4C shows an example schematic of an on-demand drug release system with a 3D structure containing a therapeutic agent (left) before a release of the therapeutic agent via application of NIR and after the release (right) of the therapeutic agent.

FIGS. 5A-5E show characterizations of NIR sensitive 4D printed constructs. FIG. 5A shows effect of the parameters of a NIR laser and material components on the 4D transformation process, including different graphene content, exposure distance, and laser intensity. The plots with squares, □, (6 cm, 8 cm, 10 cm) represent 800 mW NIR illumination; the plots with circles, ◯, (14 cm, 16 cm, 18 cm) represent 1,800 mW NIR illumination; the centimeters indicate different exposure distances. FIG. 5B shows cyclic voltammogram curves of different nanocomposites doped with 12%, 16%, and 20% graphene; the inset curve shows nanocomposite with 12% graphene. The plots illustrate different brightness of the lamp, wherein 20% sample, 16% sample, and 12% sample are shown indicating good electroconductivity of the samples. FIG. 5C shows optoelectrical property of the nanocomposites with 16% graphene; when the light is on, a slight photocurrent can be detected; after the light is turned off, the photocurrent disappears. FIG. 5D shows neural stem cell (NSC) proliferation on different 4D printed samples after 1, 3, and 7 days of culture. FIG. 5E shows images of NSC morphology on different 4D printed samples after 3 and 7 days of culture, where F-actin and nucleus are lighter shades of gray; the scale bars are 200 μm.

FIG. 6A shows 4D transformation of brain constructs from a temporary flat shape (left) to original folded shape (center) in a culture medium, when exposed to the NIR illumination. The thermal image at right shows a photothermal effect. FIG. 6B shows green fluorescent protein transfected neural stem cell (GFP-NSCs) distribution on 4D brain constructs when the flat shape of the construct (center) changed to folded shape (right); the scale bar at bottom right is 500 μm. FIG. 6C shows NSC viability (%) measurement under different photothermal temperatures when cells were exposed to NIR illumination. FIG. 6D shows fluorescent images of GFP-NSCs under different photothermal temperature; each scale bar is 200 μm. FIG. 6E shows immunofluorescent images of NSC differentiation on 4D printed nanocomposite brain construct compared to pure epoxy construct, after culturing in differentiation medium for 2 weeks; TuJ1, nuclei, GFAP, and MAP2 are each lighter gray, and the scale bars are each 200 μm.

FIG. 7 shows an example of a self-assembling molecule or self-assembling monomer (SAM) for inclusion in a matrix.

FIG. 8 shows an example of a self-assembled nanostructure, including a hydrophobic core, for inclusion in a matrix.

FIG. 9 shows load-extension curves of example nanocomposite constructs with different graphene content and pure SMP characterized at room temperature via uniaxial tensile testing.

FIG. 10 shows a flow diagram of a process for making an implantable 3D structure holding a therapeutic agent.

FIG. 11 shows a cross-sectional view of example layers of an implantable 3D structure holding a therapeutic agent.

### DETAILED DESCRIPTION

The present technology provides an on-demand drug release system including an implantable drug delivery platform with a therapeutic agent embedded in a 3D structure. The 3D structure holds the therapeutic agent until a controlled release of the therapeutic agent is initiated by absorption of a release signal. The 3D structure can include a holder for the therapeutic agent. The 3D structure includes a shape memory polymer (SMP) capable of a shape change. A receiving agent capable of absorbing the release signal can transduce the release signal and induce the shape change to release the therapeutic agent.

The release signal can be a bandwidth of electromagnetic radiation (EM). The receiving agent is configured to transduce the EM to the SMP of the holder. The EM is transduced to a sufficient heat to raise the temperature of a portion of the SMP or holder above a transition temperature range (Ttran) or a glass transition temperature (Tg) of the SMP, to cause a shape change that releases the therapeutic agent from the holder and into the subject. At least a portion of the therapeutic agent is released. After the release, the therapeutic agent can be further targeted to a specific region of the subject (e.g., a region of acute pain). As used herein, the Ttran is a temperature range including a lower temperature below the Tg and a higher temperature above the Tg, whereupon the SMP begins a shape change at either the lower temperature or the higher temperature. For example, the Ttran can be about Tg±5° C., about Tg±4° C., about Tg±3° C., about Tg±2° C., about Tg±1° C., depending upon the SMP used. A Ttran can begin to initiate a shape change in the SMP without reaching the Tg.

The therapeutic agent can include a matrix between the therapeutic agent and the SMP or the holder. The matrix can include or consist of a biodegradable polymer that can encapsulate the therapeutic agent or can be associated with the therapeutic agent or both. As used herein, the term “associated with” includes hydrogen bonding, ionic bonding, metallic bonding, polarized bonding, van der Waals bonding, π-π interactions, cation-π interactions, clathrate bonding, a physical entrapment, a confinement, or a combination thereof. A confinement can be in lamellar microstructure or in pores of the SMP.

The biodegradable polymer can include a targeting moiety that delivers the therapeutic agent to a specific region of the subject. The term “biodegradable polymer” means a polymer or a formulation that is capable of at least a partial excretion from the subject. The biodegradable polymer can be physically entrapped in the SMP of the 3D structure, can be associated with the SMP, can be held by lamellar microstructures or pores of the SMP, or can be contained in the SMP. The biodegradable polymer can include or consist of a self-assembled biodegradable polymer (SABP) or a self-assembling monomer (SAM). The matrix can include additional additives, formulations, polymers, elements, targeting moieties, or a combination thereof.

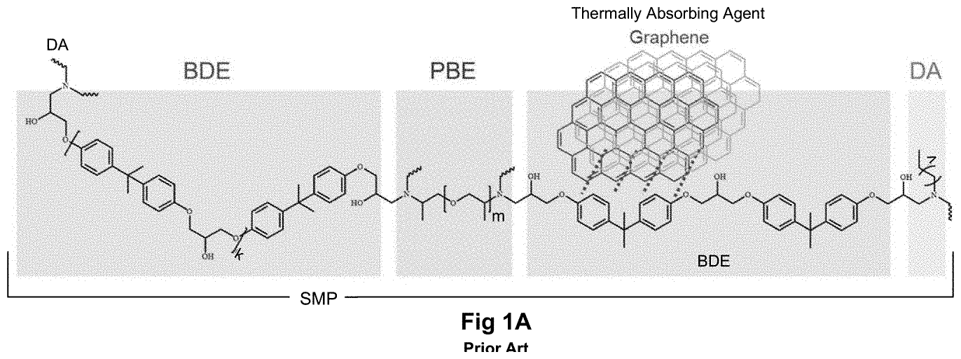

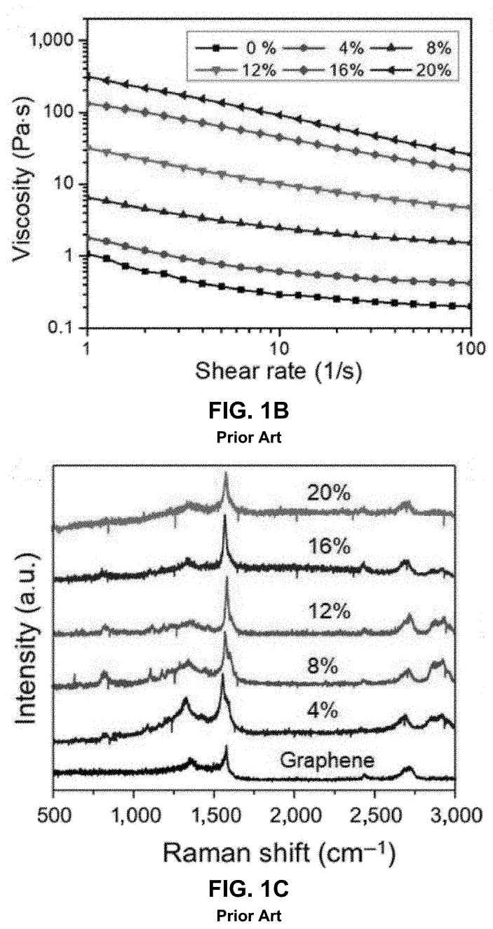

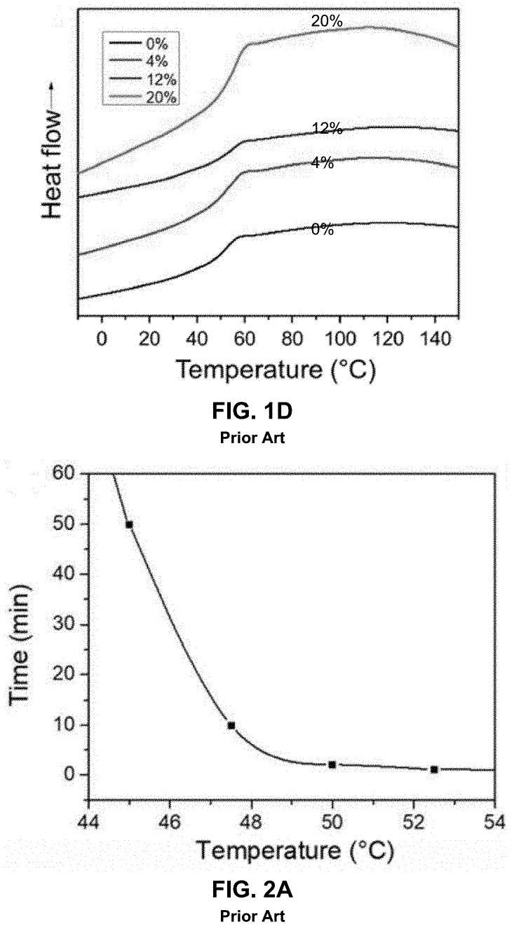

An example chemical structure of a monomer of a SMP is depicted in FIG. 1A. In FIG. 1A, the graphene is depicted with π-π interactions or van der Waals forces (dotted lines) to the SMP. The graphene depicts an example of a receiving agent capable of absorbing NIR radiation and transducing heat energy to the SMP. The temperature of at least a portion of the SMP can be raised to Ttran or to greater than the Tg, thereby initiating a shape change in at least a portion of the 3D structure.

The example SMP includes an aliphatic diamine crosslinker (poly(propylene glycol) bis(2-aminopropyl) ether), (PBE). The variable “m” shown in the PBE can be an integer in the range from 1 to 4.

At the left and right of the PBE, the SMP includes a rigid epoxy monomer (bisphenol A diglycidyl ether), (BDE), and a crosslinking modulator shown at both ends of the monomer (decylamine, DA) which can be used to tailor the Tg of the assembled polymers. The variable “k” shown in the BDE can be an integer in the range from 1 to 2. The variable “v” shown in the DA is 8 for DA.

Different formulations can be optimized to obtain the SMP with Tg at about 45° C. (Xie, T., Rousseau, I. A. 2009). The Tg can be slightly greater than 37° C., ensuring an effective shape fixation at a cell culture temperature or at normal human body temperature. For example, the Tg can be a temperature in the range from about 38° C. to about 90° C., in the range from about 39° C. to about 60° C., in the range from about 40° C. to about 55° C., in the range from about 42° C. to about 50° C., in the range about 43° C. to about 48° C., or in the range from about 44° C. to about 46° C. The Tg of a SMP used herein can be greater than about 37° C., greater than about 38° C., greater than about 39° C., greater than about 40° C., greater than about 41° C., greater than about 42° C., greater than about 43° C., greater than about 44° C., greater than about 45° C., greater than about 46° C., greater than about 47° C., greater than about 48° C., greater than about 49° C., or greater than about 50° C. For a Tg at about 45° C., an optimal molar ratio of the composition can be 0.01 mol BDE, 0.003 mol PBE, and 0.004 mol DA. The mixture of BDE, PBE, and DA can be referred to as an epoxy ink system for 3D printing. Graphene nanoplatelets (e.g., about 6-8 nm thick×5 μm wide) at different concentrations can be dispersed into the epoxy ink system without requiring the use of any solvent. The effect of mechanical stirring, sonication time, storage time, and graphene concentration on the dispersion and reaggregation of graphene in epoxy mixtures is analyzed in various studies (Wei, J. C., et al., 2015). Due to the strong van der Waals force (high viscosity) of ink and the π-π stacking between rigid aromatic epoxy and dispersed graphene (FIG. 1A), graphene can be homogeneously dispersed in the ink without a pronounced tendency of reaggregation.

Other shape-memory polymers can be for an implantable SMP with a Tg slightly higher than 37° C., for example, polylactide (PLA).

To study the printability of the nano-composite inks in an extrusion system, the dynamic viscosity is measured with a rheometer at room temperature. FIG. 1B shows nanocomposite inks with varying amounts of graphene have a typical shear thinning behavior, and the viscosity of the inks increases with increasing graphene content. In FIG. 1B, 0% (wt/wt), 4%, 8%, 12%, 16%, and 20% graphene contents are compared. The 0% graphene (wt/wt) ink at bottom has the lowest viscosity. It is found that it is hard to extrude the ink in a custom printing system when the graphene concentration is higher than 20%. Therefore, the nanocomposite inks with 0%, 4%, 8%, 12%, 16% and 20% graphene are systematically investigated herein. In a reported study, printed high-content graphene scaffolds have been developed, indicating the high content graphene (>60%) possesses good biocompatibility, which is verified by in vitro and in vivo studies (Jakus, A. E., et al., 2015).

FIG. 1C shows Raman spectra of nanocomposites including various weight percent of graphene (20%, 16%, 12%, 8%, 4%), with graphene shown at bottom. Three prominent peaks of graphene nanoplatelets are ˜1,350 cm⁻¹ (D-mode), ˜1,580 cm⁻¹ (G-mode), and ˜2,710 cm⁻¹ (2D-mode). The D-mode appears at approximately 1,350 cm⁻¹; the G-mode is located around 1,580 cm⁻¹; and the 2D-mode peak is at approximately 2,710 cm⁻¹; which are the three prominent peaks of graphene nanoplatelets. With different graphene content, some new peaks appear around 820, 1,100, 2,850, and 2,950 cm⁻¹, which indicate the intermolecular interactions of the aromatic epoxy/graphene and graphene/graphene in epoxy.

FIG. 1D shows DSC curves of nanocomposites including 4%, 12%, and 20% graphene and pure SMP (0%, bottom). The DSC data indicate these nanostructures have a similar Tg of about 45° C. There is not any significant change observed when varying the graphene content. No melting peak is observed, indicating the SMPs are highly cross-linked and have no crystalline domains. In the glass phase (lower than Tg), the material is rigid and cannot be bent easily. While the temperature increases beyond Tg, the material enters the soft rubber phase and its malleability increases. The reversible transition between these two phases of SMPs (glass and rubber) results in a sequential shape memory cycle. In FIG. 1D, the arrow at left indicates the endothermic process.

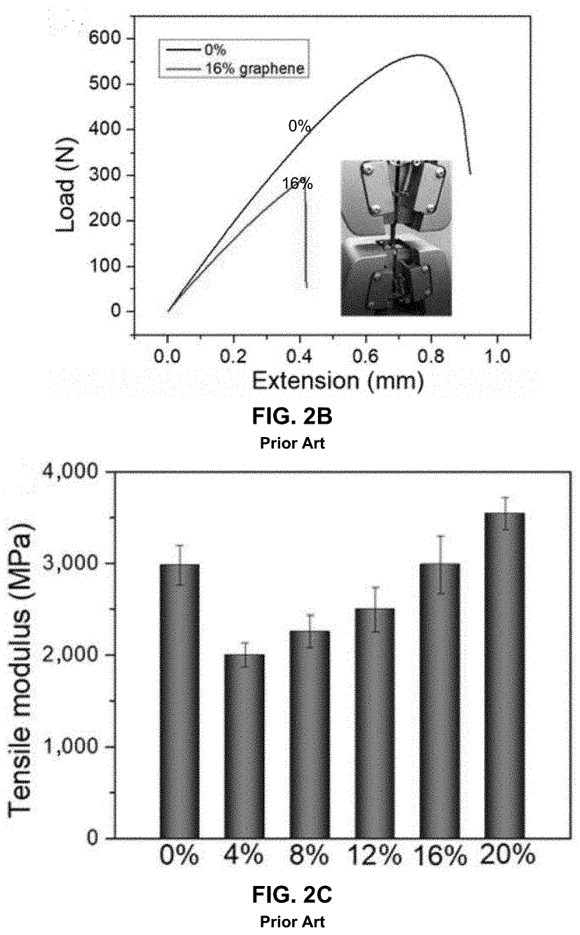

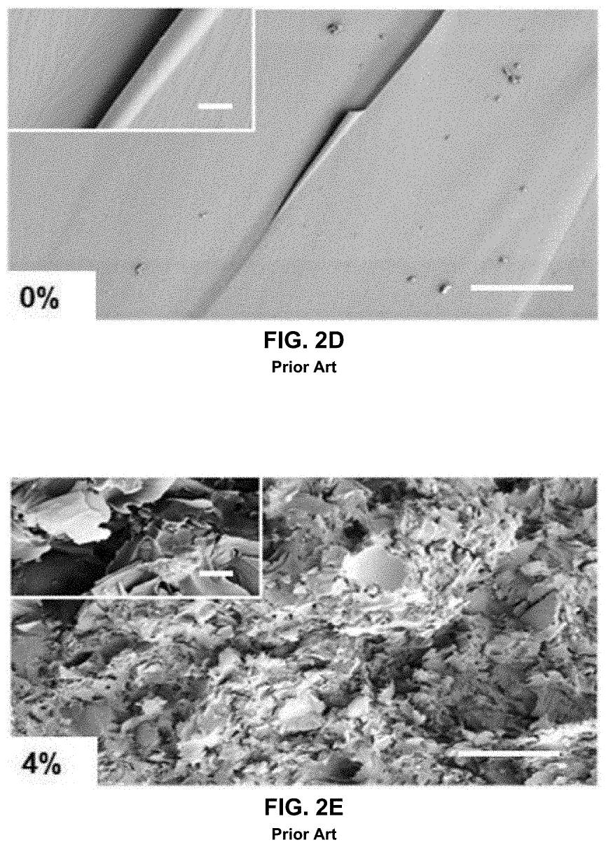

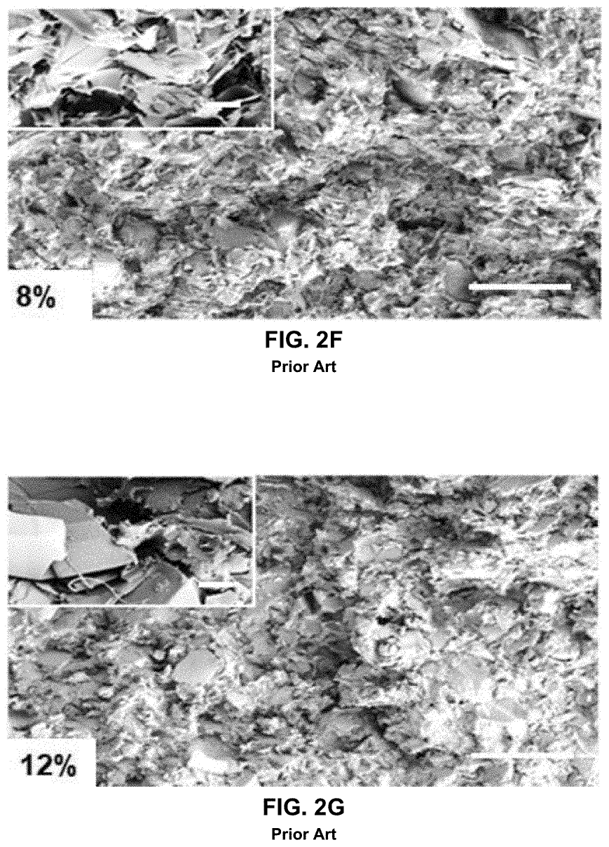

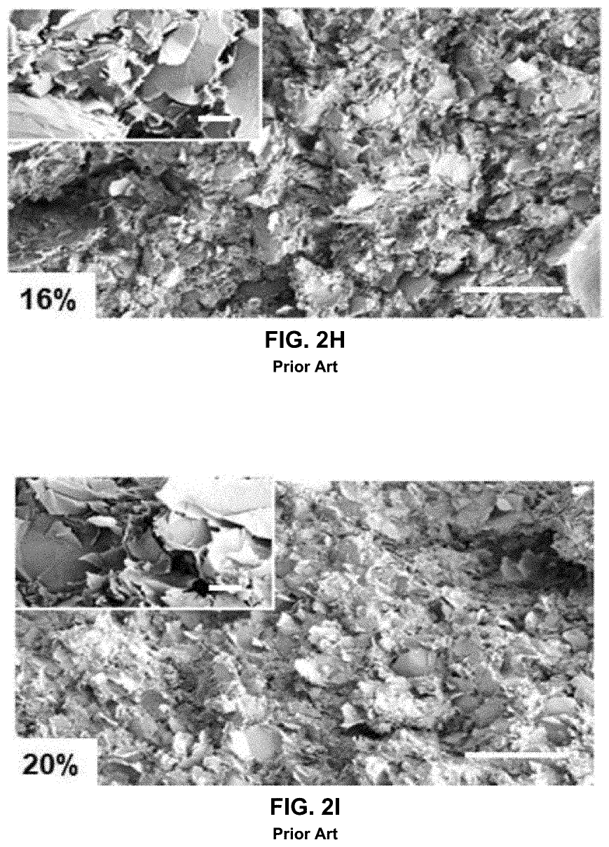

To study the thermosensitive shape memory effect, the printed nanocomposite samples, having an initial permanent shape, are bent into a U shape at 60° C. (above Tg) to a temporary shape using an external force, and the temporary shape is kept at room temperature (below Tg). After cooling the temporary shape below Tg, the temporary shape is constant without application of the external force. When the samples are placed in a thermostat (e.g., the external temperature or triggering temperature is above Tg), they recover their initial and permanent shape over time. The strain recovery rate (Rr) is the ability of the material to recover to its permanent shape, and the ability of the switching segments to hold the applied mechanical deformation is called the strain fixity rate (Rf). After the calculation, the samples exhibit an Rf of −99%, and an Rr of −95%, suggesting the samples almost return to their full original shape. The addition of graphene does not affect the original shape memory behaviors of the SMP (an Rf of −99%, and an Rr of −95%). Additionally, the shape-changing (transformation) duration is distinctly different by varying the triggering temperature. As shown in FIG. 2A, the transformation time considerably decreases with increasing triggering temperature.

The mechanical behavior of the nanocomposites is characterized at room temperature via uniaxial tensile testing, and the results are shown in FIG. 2B and FIG. 2C. Details of tensile curves are also shown in FIG. 9. Compared to pure material (0%), the nanocomposites have a lower modulus, indicating the doping of graphene can negatively affect the intrinsic structure of the pure epoxy. The tensile modulus of the nanocomposites increases with increasing graphene content, indicating the reinforcement effects of the graphene in the epoxy. Additionally, the nanocomposite doped with 16% graphene shows a similar tensile modulus value with the pure material. However, the extension value of the nanocomposite is significantly lower. This suggests that the doping of graphene increases the material's rigidity while depreciating its malleability.

The microstructure characteristics of the epoxy and its nanocomposites are studied using SEM as shown in FIGS. 2D-2I. The cross-sectional images show that pure epoxy with 0% graphene (FIG. 2D) had a smooth, fractured surface while graphene doping led to lamellar microstructures (FIGS. 2E-2I). Therefore, it is deduced that the different microstructures contribute to the large difference between mechanical moduli. After the porous structure was introduced, the tensile moduli decreased compared to pure material. With increasing graphene content, a denser lamellar arrangement increases the material's rigidity.

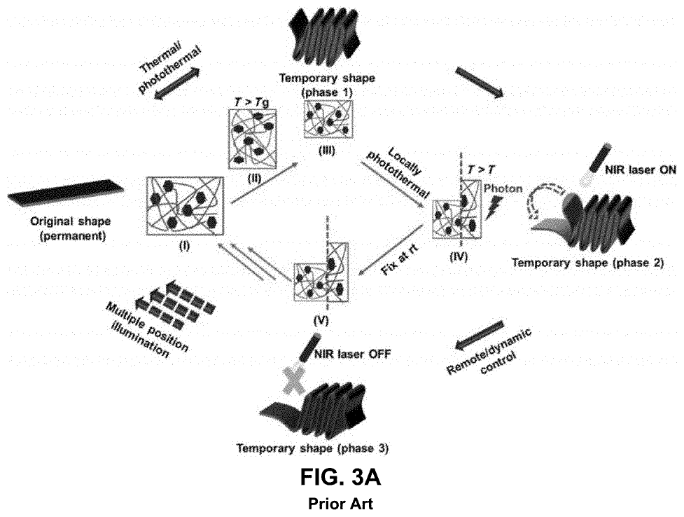

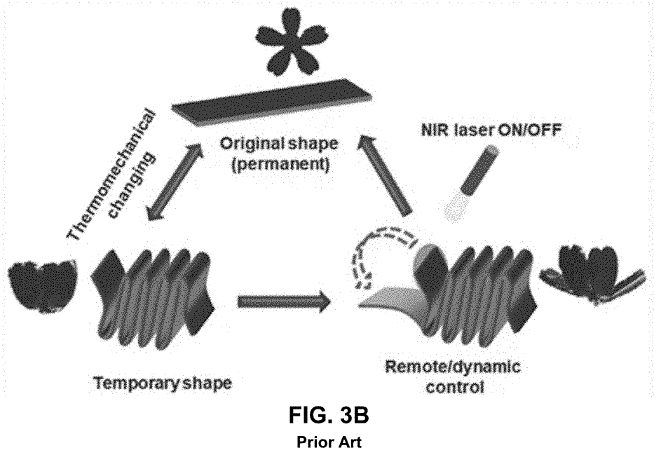

FIG. 3A shows a schematic of the dynamically controllable transformation of the 4D printed NIR sensitive SMPs. After printing, the nanocomposite construct has an initial (permanent) shape shown at left at room temperature (I). The nanocomposite construct is exposed to NIR illumination for heating (a photothermal-triggered process) or directly heated (a conventional thermal-triggered process) to a temperature above the transition temperature (or Tg). A stress is applied at T>Tg, where the construct's shape is changed by an external force to a temporary shape (II and III). The temperature of the construct is decreased below Tg, and then the external force is removed. At this stage, the construct's shape is changed and fixed (III) at room temperature (Phase 1, Temporary shape). The Phase I, Temporary shape at the top of FIG. 3A can be an implantable shape with a therapeutic agent held by the 3D structure. Once local reheating up to the Tg through NIR exposure is applied (right), the temporary shape of the construct can gradually return to its original shape (III→IV→V→→→I). During this photothermal process, the NIR illumination is able to remotely control the shape-changing position and transformation time of the nanocomposite constructs (Phase 2→Phase 3).

In FIG. 3B, an original shape (top) can be formed by 3D-printing or by other methods. In FIG. 3B, the original shape (top) is formed into a temporary shape (bottom-left) by an external force such as an external mold or pressure, which can be gradually returned (i.e., part-by-part, bottom-right) to original shape via remote/dynamic control, such as through application of NIR laser. Multiple position illumination can return the shape at bottom-right to the original shape at top.

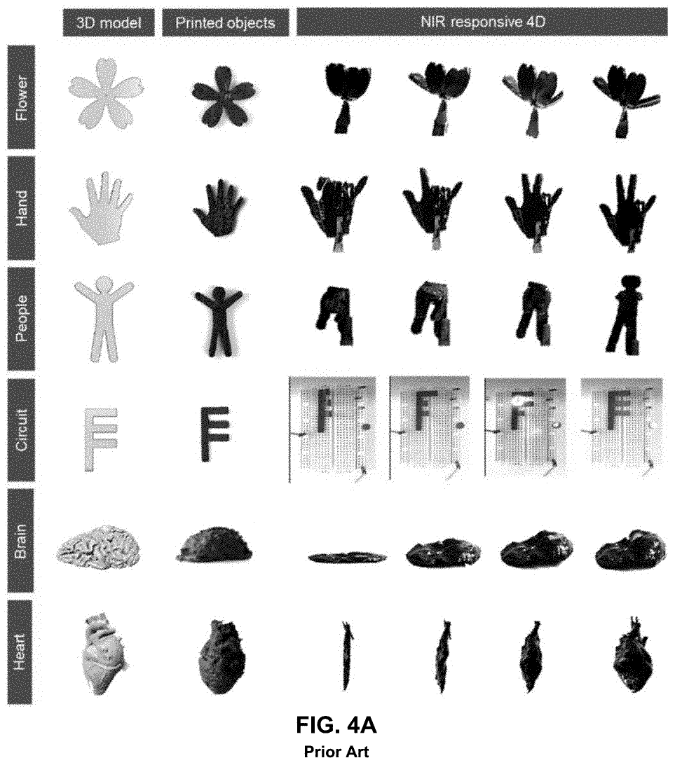

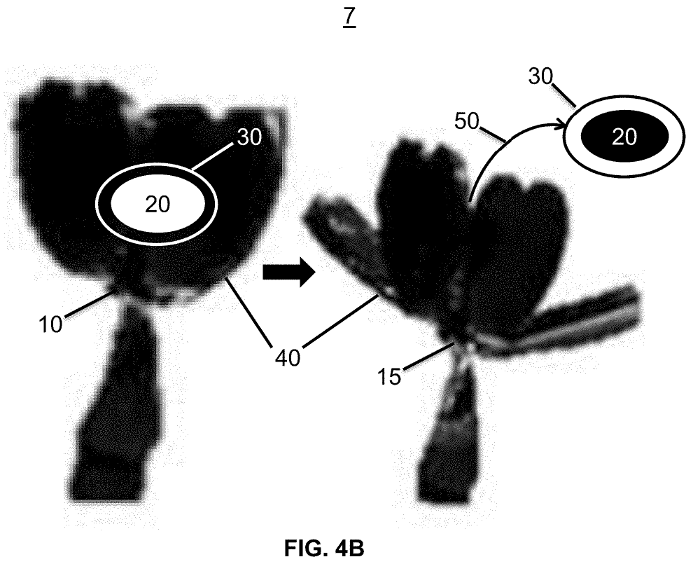

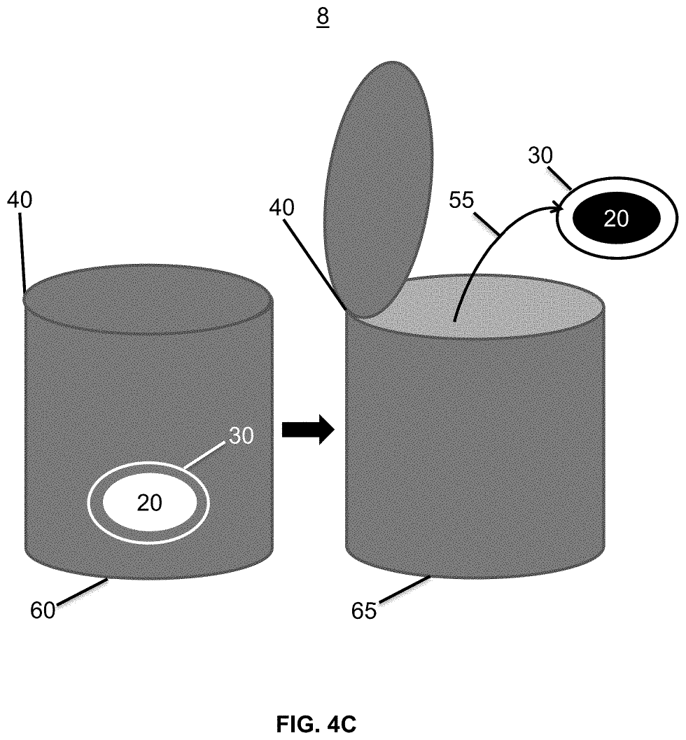

FIG. 4A shows images of the NIR sensitive 4D transformation behavior of various nanocomposite models in response to directed NIR radiation, which is different from the uncontrollable shape changing in a thermal-triggered (applying heat over the entire structure) 4D changing process. In FIG. 4A, various models are 4D printed, including a blooming flower, hand gesture, exerciser, controllable circuit switch, folded brain, and dilated heart. The printed objects shown in FIG. 4A are fixed to a temporary shape by application of an external force above the Tg and then by cooling to below about 37° C. The temporary shape is shown at the left of the “NIR responsive 4D” column. After the temporary shape is fixed, the 4D printed models are heated at various specific locations to gradually recover the original shape. Applying heat over the entire structure causes a traditional “thermal-triggered” recovery process to the originally printed shape and is not easy to control, so local heating can be applied (via NIR laser) to accurately control shape transitions. Comparatively, the “photothermal-triggered” transformation exhibits a precisely and conveniently controllable feature.

FIG. 4B shows an example of an on-demand drug release system 7 with a temporary shape 3D structure 10 (left) holding a therapeutic agent 20 associated with a biodegradable polymer 30. Application of NIR radiation 40 on the temporary shape 10 is thermally absorbed by the receiving agent of the SMP and causes a gradual shape change 15 to the permanent shape and at least a partial release 50 of the therapeutic agent 20 and biodegradable polymer 30. In FIG. 4B, the biodegradable polymer 30 is optional. Structures 10 and 15 are implanted in a subject. The application of NIR radiation 40 can be applied by a health care provider external to the subject. The therapeutic agent or the biodegradable polymer can be associated with the 3D structure or in a confinement in lamellar microstructure or in pores of the SMP. In FIG. 4C, an on-demand drug release system 8 with a holder in a temporary shape 60 (left) contains a therapeutic agent 20 and biodegradable polymer 30. Application of NIR 40 causes a shape change in at least a portion of the holder to permanent shape 65 (right). After the shape change at right, there is at least a partial release 55 of the therapeutic agent 20 and biodegradable polymer 30 in a subject.

As depicted in FIGS. 4A-4B, after forming a temporary shape around a therapeutic agent, when exposed to a NIR laser, these 4D printed models experience a gradual, targeted shape change, where the time and position of the transformation is precisely controlled by the position of the NIR exposure. It is observed in FIG. 4A that the architectural detail of the constructs such as the petals, fingers, were able to be remotely, locally, and precisely controlled without a complicated predesign. In FIG. 4A, the circuit image also illustrates the brightness of light gradually increases by dynamically controlling the connection of the circuit switch, which suggests its great potential for developing intelligent circuitries or robots.

Graphene can absorb photons of NIR radiation, resulting in a conformation change or shape change. The shape change can initiate at Ttran. Above the glass transition temperature (Tg) of a SMP, a printed object transforms its shape via a “thermomechanical reprogramming” process when irradiated with NIR radiation. Irradiating with light-switch activated stimulation can achieve a remote, precise, and dynamic control of both time and position. Long-wavelength NIR is regarded as a biologically benign energy form capable of efficiently penetrating tissue with no biological harm when compared to other energy sources. The shapes designed from the 3D printing can be designed in nearly any shape and can be designed to provide remotely and dynamically controllable 3D components at any area of the shape.

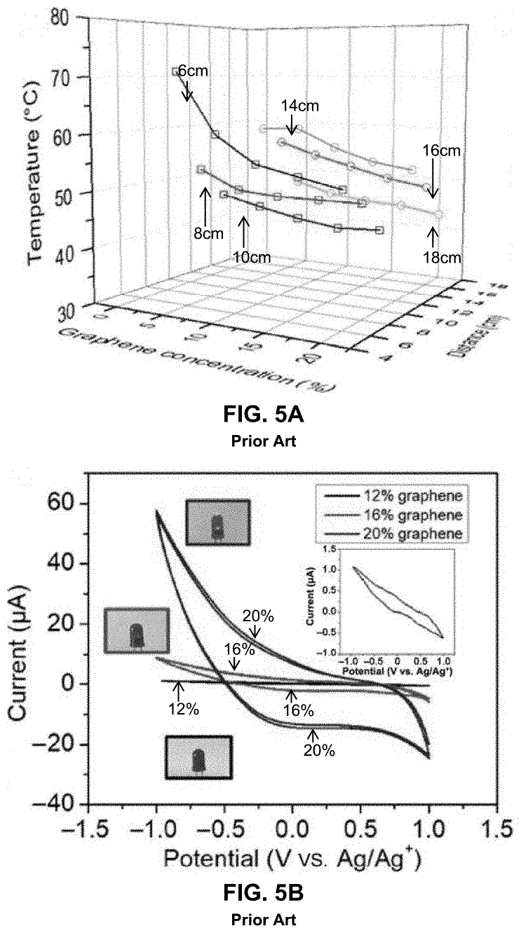

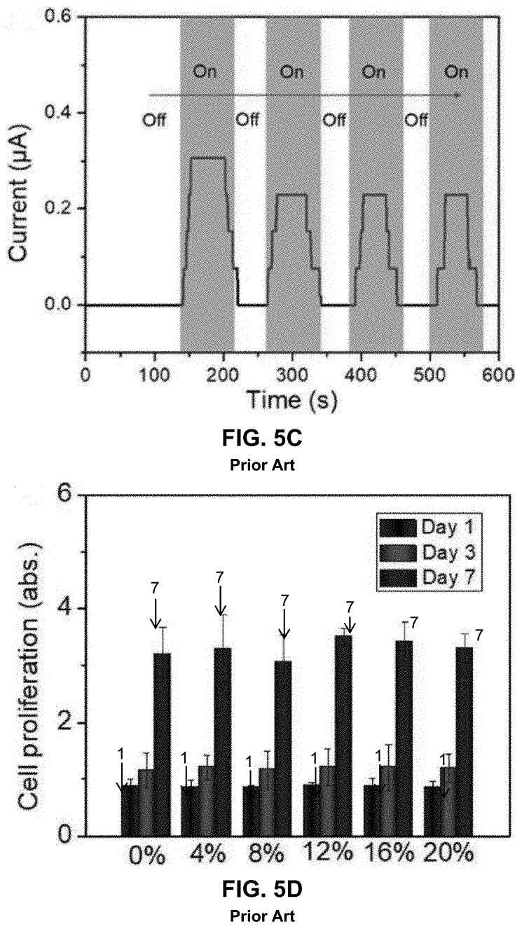

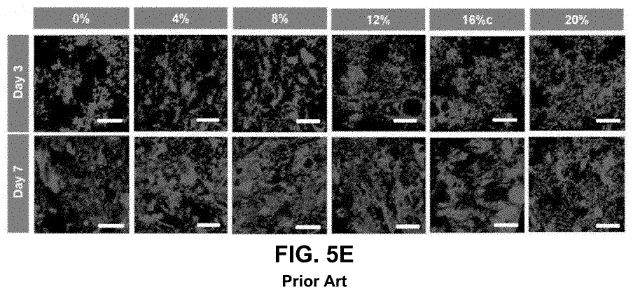

To further explore the effect of the parameters of the NIR laser and the material components on the 4D transformation process, different nanocomposites, exposure distance, and laser intensity are systematically studied as shown in FIG. 5A. The Tg decreases with increasing graphene content for NIR exposure at either 800 or 1,800 mW, and the low laser intensity results in a more distinct decrease. This phenomenon is due to the faster heat dissipation for nanocomposites with a higher graphene content (higher thermal conductivity) when considering a dynamic balance between “photothermal triggered” heating process and heat dissipation. To obtain a similar photothermal temperature, a shorter exposure distance is applied for the NIR exposure with low laser intensity. This means a higher laser intensity is able to achieve the remote control of 4D transformation, which is an additional advantage for NIR responsive 4D printing. Moreover, the nanocomposites with a lower graphene content (0.5%) also exhibit an excellent photothermal effect (not shown), but pure epoxy kept a constant temperature (room temperature) in any case.

As the graphene nanoplatelets are applied in these designs, the resulting electroconductive and optoelectronic properties of the nanocomposites are important physical characteristics. It is expected that these unique features of nanocomposites could improve cellular functions, including signal transmission of neural cells and the autonomous beating of cardiomyocytes for electroactive tissue regeneration applications (Cui, H. T., et al., 2014; Zhu, W., et al., 2018). FIG. 5B shows cyclic voltammograms (CVs) that characterize the redox properties of nanocomposites. The results show that 4D printed nano-composites doped with 12%, 16%, and 20% graphene undergo reversible redox reactions, where the enclosed area of one CV cycle is proportional to the charge storage capacity (Zhu, W., et al., 2018; Cui, H. T., et al., 2014; Cui, H. T., et al., 2013). There is no curve observed for other samples with lower graphene content, suggesting their non-electroactivity or nonconductivity. Similar conductivity results are also confirmed by the 4-Point sheet resistance testing. The conductivities of nanocomposites with 12%, 16%, and 20% graphene are ˜5×10⁻⁵, 1.27×10⁻⁴, 5.32×10⁻⁴ S/m, respectively, which are within the electroconductivity range of conductors.

Other samples were nonconductive (lower than 1×10⁻⁶ S/m). Moreover, a slight and damped photocurrent is detected when the nanocomposite is exposed to NIR illumination (FIG. 5C). This trial indicates the nanocomposite can produce and deliver charge under light illumination, although its performance might not reach the qualification of other photocurrent devices. Bioelectricity plays an essential role in the functioning of all living organisms, not just in the action potentials of nerves and muscles, but also in regulating cellular functions. In the future, it is expected that a perfect tissue construct with high optoelectronic conversion efficiency can be created to produce higher charge density, which is able to improve the electroactivity of engineered tissue without the need for a complicated stimulation device.

Additionally, cell proliferation and morphology are evaluated, where NE-4C neural stem cells (NSCs) are seeded on the different printed samples (FIG. 5D and FIG. 5E). After 7 days of culture, there is no significant difference among pure epoxy and nanocomposites by varying the doped graphene content. All samples exhibit excellent cell spreading morphology identified by F-actin staining. This suggests that although a high graphene content is used in the nanocomposites, the materials exhibit an excellent cytocompatibility.

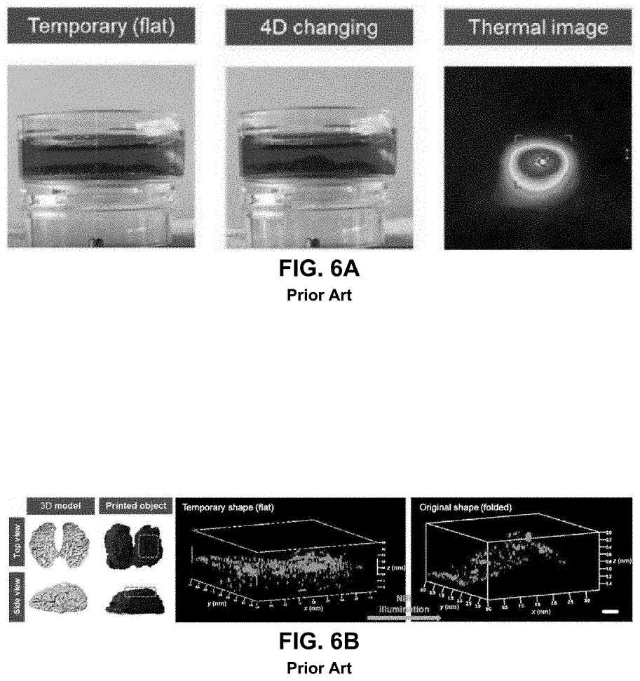

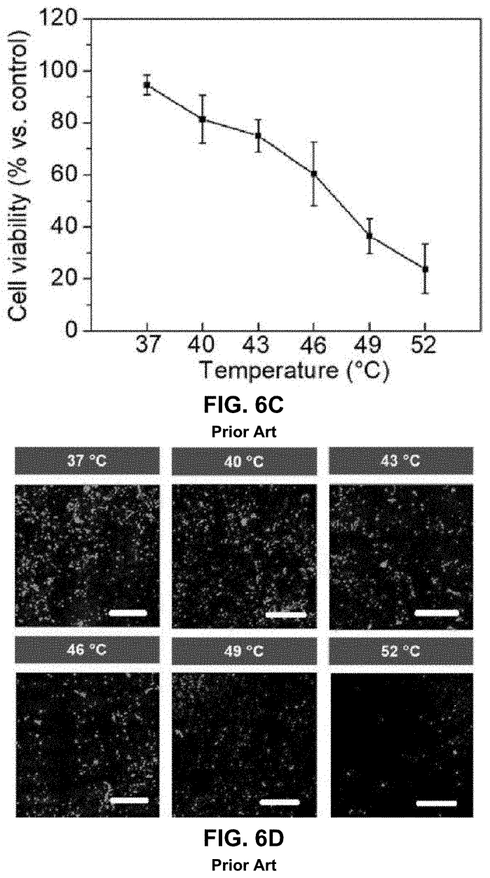

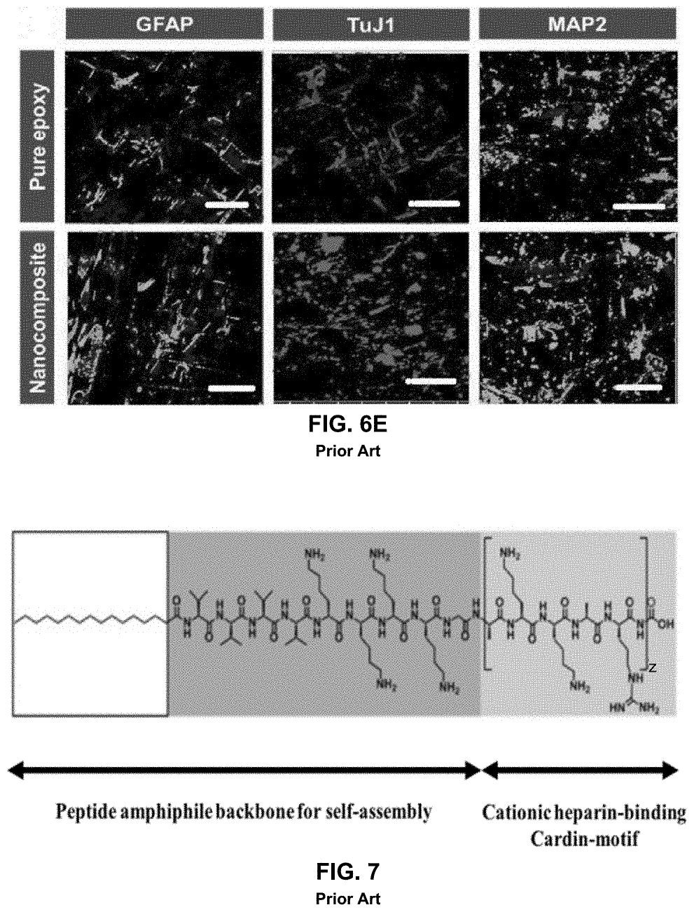

Considering an appropriate printability, high mechanical strength, conductivity, and cell growth, a 16% nanocomposite ink is selected to conduct additional studies. A 4D printed brain model was designed, and NSCs are utilized to create the neural tissue construct. The brain constructs incubated in culture medium exhibit an excellent “shape fixation-NIR triggered 4D recovery” process as shown in FIG. 6A. After creating the tissue construct, the 3D construct was temporarily fixed to a flat shape. Then NSCs were seeded onto the flat surface of the construct and further cultured for several days. After experiencing the neural differentiation, the cell-laden construct was mildly exposed to NIR illumination for recovering the original 3D brain shape. The thermal image at the right of FIG. 6A shows the exposure region of NIR laser had a high and focused temperature distribution on the 4D printed construct.

Owing largely to gravity, cells immediately exhibited a non-uniform distribution and migrated to lower areas prior to adhesion. FIG. 6B shows fluorescent images of the NSC distribution on the 4D brain construct by confocal microscopy when the temporary flat shape of construct changed to the original folded brain-like shape under NIR exposure. It indicates the 4D printed construct is able to obtain a uniform cell distribution on the complex 3D architecture after shape transformation. To further investigate the cell viability under photothermal transformation process, different photothermal temperatures are applied, and the results are shown in FIG. 6C and FIG. 6D. With increasing temperature, a significant decrease in cell viability is observed. Fluorescent microscopy images showed the number of green fluorescent protein transfected NSCs (GFP-NSCs) decreased after light exposure. However, above the Tg of 4D transformation, cell viability (%) is higher than 60%, ensuring sufficient cell number on the engineered tissue construct after photothermal transformation.

Furthermore, a NSC differentiation study was performed after 2 weeks of differentiation medium culture, and results are shown in FIG. 6E. During the differentiation culture, intermittent NIR illumination was applied to the nano-composite construct to achieve a 4D transformation associating with an optoelectronic stimulation. The immunofluorescent images illustrate obvious differentiated neurons identified by neuron-specific Class III β-tubulin (TuJ1) and microtubule-associated protein 2 (MAP2), and some astrocytes detected by glial fibrillary acidic protein (GFAP). TuJ1 was expressed in newly generated immature neurons; when a mature neuron was generated, involving in microtubule assembly, MAP2 was detected as an essential step in neurite formation (Zhou, X., et al., 2018).

Additionally, star-shaped glial cells in the brain known as astrocytes are also found. Compared to a pure epoxy construct, a higher expression of the neurogenic protein was observed on the nanocomposite construct. It is hypothesized the bioelectricity can improve the neurogenic differentiation during the culture period. In general, the 4D printed nanocomposite construct exhibits a high potential in neural engineering. When taking in vivo implantation into consideration, the lowest possible graphene and the use of biodegradable polymer can be utilized. More importantly, the current technology can successfully create an in vitro 4D printed organ model to achieve a novel concept about NIR responsive 4D printing, further illustrating the advantages of the 4D transformation system, e.g., ease of operation, high biosafety, NIR sensitivity, remote and dynamic control as well as spatiotemporal synergy. This novel 4D printing technique also has great potential for other applications, such as intelligent robots and controllable circuits.

The smart epoxy with a shape memory property can be synthesized, and its graphene doped nanocomposites exhibit an excellent photothermal effect. Compared to other 4D printed materials, the NIR responsive 4D printed nanocomposite possesses a dynamically and remotely controllable transformation in a spatiotemporal manner. The 4D printed brain construct provides a facile method for fabricating a dynamic tissue construct to satisfy demands on structures and functions. By combining with its electroconductive and optoelectronic properties, the 4D neural cell-laden construct exhibits excellent neural stem cell growth and differentiation.

The SMP technology can be used in a variety of ways. Assembled nanomaterials, such as nanocomposites or nanostructures (for example, particles, films, rods, stars, tubes, and platelets) containing graphene as an electrically active component can be used to stimulate functions of cells, such as neurons, cardiovascular cells, osteoblasts, and chondrocytes. The printed shapes can conduct nerve impulses. Such electrically active nanostructures also can be used to provide dynamic control in response to NIR light over release of bound molecules, such as therapeutic agents.

The materials and structures of the present technology can be used to provide constructs for replacement or regeneration of tissues and organs, such as cartilage, vascular tissue or parts of the vascular system, heart, brain, or spinal cord. The materials and structures of the present technology can contribute to improved therapies for treating neurological diseases, for example, Alzheimer's disease, Parkinson's disease, and rare diseases. Use of the materials and structures can be coupled with pain medication therapy, opioid and non-opioid analgesics, for use without risk or with reduced risk of addiction.

The novel materials and structures of the present technology can be used in the discovery and development of new pharmaceutical agents for disease prevention, diagnosis, and treatment. Further, they can be used to develop a new generation of implantable medical devices. For example, an implanted structure can contribute to the autonomous beating of cardiomyocytes and eliminate the need for a pacemaker. An implanted structure can be used to promote electroactive tissue regeneration and to prevent further myocardial damage after myocardial infarction. The materials and structures herein are useful in orthopedic applications, for example by serving as orthopedic bone screws that simultaneously provide analgesia in combination with opioid and newly developed non-opioid analgesics. Further, the materials and devices of the present technology can be used to develop intelligent circuitries coupled with artificial intelligence for improving electroactivity of engineered tissue without need for a stimulation device.

Such SMP nanomaterials can be used to deliver molecules or therapeutic agents under encapsulation procedures for later release in the body of a subject. Encapsulation of molecules or agents can include a biodegradable polymer or a SABP between the SNP and the therapeutic agent. The molecules or therapeutic agents can be further encapsulated by a different polymer, for example, in nano-structure, micro-structure, or larger capsules, including a polymer different than the biodegradable polymer. For example, the therapeutic agents discussed herein can be nano-encapsulated, micro-encapsulated, or encapsulated in a formulation polymer or formulation coating. Examples of formulation polymers or coatings include N-(2-hydroxypropyl)methacrylamide (HPMA), liposomes, alginates, PEG, poly(glutamic acid), polyethylenimine, dextran, dextrin, chitosans, poly(l-lysine), and poly(aspartamides).

The technology provides methods for administering a therapeutic agent to a subject. In an example, a method can include one or more steps of: providing a therapeutic agent associated with a SAM; embedding the SAM in a 3D printed structure including a shape memory polymer and graphene; implanting the 3D printed structure in a subject; directing a NIR signal towards the subject's skin such that the signal penetrates the subject's skin and is at least partially absorbed by the 3D printed structure, the signal causing the structure to release the therapeutic agent associated with the embedded SAM. The embedding can be at any point of the method.

A method of NIR excitation release of a therapeutic agent in a subject can include shining NIR upon a subject with the NIR directed to a 3D printed structure (e.g., on-demand drug release system), which has been implanted within the subject. The 3D printed structure can include a matrix with biodegradable polymer or a SABP embedded in the matrix. A polymer can encapsulate or can be associated with a therapeutic agent. The polymer can be designed to target an area within the subject's body (after release from the on-demand drug release system or 3D printed structure). The area can be nearby the 3D printed structure, or the area can be distant. The method can be used for NIR release of any suitable therapeutic agent, for example, an analgesic. The method can include a NIR light excitation release of drugs with the wavelength specific to a NIR wand or NIR emitter only accessible by a health care provider. The method can prevent abuse of analgesics by controlling release such that pain is managed more effectively. The polymer can be bound or associated with the SMP, or with lamellar microstructures or pores of the SMP, or embedded in pores or lamellar microstructures of the SMP.

The ability to transform the custom materials utilizing a NIR signal applied from outside a subject, combined with the excellent cytocompatibility of the custom materials demonstrates unexplored drug delivery capabilities. For example, a therapeutic agent can be combined with, encapsulated in, or associated with a biodegradable polymer, a SABP, or a self-assembled molecular delivery system. The polymer, along with the therapeutic agent, can be released by the custom materials. For example, the 3D printed structure can include a matrix with a biodegradable polymer or a SABP embedded in the matrix.

Biodegradable polymers can include polyanhydrides, polyphosphazenes, poly(orthoesters), polyesters, polysaccharides including chitosans, alginates, and celluloses, proteins, microbially synthesized polymers, polycarbonates, polycyanoacrylates, polyamides, polyurethane, polyphosphoesters, and examples include chemical polymerizations of biomonomers such as polylactide, polycaprolactone, polybutylene succinate, polybutylene succinate adipate, aliphatic-aromatic copolyesters, polybutylene adipate/terephthalate, and polymethylene adipate/terephthalate. The biodegradable polymers or the self-assembling biodegradable polymers can include SAMs (self-assembled monomers). A SAM can include various sequences. For example, the methods herein can be practiced with a SAM including SEQ ID NO: 1 shown below:

### FA-A1-A2-A3-A4-A5-A6-A7-A8-A9-[A10-A11-A12-A13-A14-A15]n-Q (SEQ ID NO: 1),

wherein Q is not present, is —H, is —N(C═O)OH, or is —COOH; wherein FA is a fatty acid, an alkyl, or alkenyl forming an amide linkage, amide bond, or a direct —CH₂—N— bond to nitrogen (—N—) of A1; A1-A8 and A10-A14 are amino acids; A9 is not present, is a bond, or is an amino acid; and A15 is not present or is glycine (G), preferably each amino acid independently selected from naturally occurring amino acids; n is 1 to 15. An example is depicted in FIG. 7. In SEQ ID NO: 1, a lipophilic amino acid can be used at A1, A2, A3, A4. A hydrophilic amino acid can be used at A5, A6, A7, A8. An optional linker amino acid can be included at A9. The A10, A11, A12, A13 can include hydrophilic amino acids, while A14 can be a cationic amino acid. A14 can be a polar residue and A10-A13 can include two cationic residues. The fatty acid can have from 2 to 30 carbons. For example, the fatty acid can be selected from the group consisting of palmitic acid, caprylic acid, capric acid, lauric acid, myristic acid, stearic acid, arachidic acid, behenic acid, lignoceric acid, cerotic acid, myristoleic acid, palmitoleic acid, sapienic acid, oleic acid, and elaidic acid.

A SAM can be SEQ ID NO: 2 or SEQ ID NO: 11,
shown below:
(SEQ ID NO: 2)
FA—V—V—V—V—K—K—K—K—G—[A—K—K—A—R]n—Q,
or
(SEQ ID NO: 11)
C₁₅—(C═O)—V₄K₄G(AKKAR)₂—Q;

wherein FA is a fatty acid forming an amide linkage with V; V is valine; K is lysine; G is glycine; A is alanine; R is arginine; n is 1 to 15; and Q is —N(C═O)OH.

A SAM can have Formula I shown below:

FA¹-S¹—C²-Q (Formula I);

wherein FA¹ is a fatty acid, alkyl, or alkenyl with an amide bond or a direct —CH₂—N— bond to nitrogen (—N—) of S¹; C² is a cationic heparin-binding (Cardin-) motif peptide; and Q is not present, —H, is —N(C═O)OH, or —COOH. FA¹ can include from 2 to 30 carbons. FA¹ can be selected from the group consisting of palmitic acid, caprylic acid, capric acid, lauric acid, myristic acid, stearic acid, arachidic acid, behenic acid, lignoceric acid, cerotic acid, myristoleic acid, palmitoleic acid, sapienic acid, oleic acid, and elaidic acid. FA¹ can be selected from the group consisting of C₁₀, C₁₂, C₁₄, C₁₆, C16:1, C₁₈, C18:1, C18:2, C18:3, C₂₀, C20:1, C20:4, C20:5, C₂₂, C22:1, and C22:6.

S¹ can have SEQ ID NO: 3, which is —(X_{N})—(Z_{N})—B³; wherein independently and for each occurrence, each of X is I, L, or V, and each Z is K or R. Each N is independently an integer selected from the group consisting of 2, 3, 4, 5, 6, 7, 8, 9, and 10. The B³ is not present, is A, or is G. In an example, S¹ can be SEQ ID NO: 4 (V₄K₄) or V₄K₄G (SEQ ID NO: 5).

A cationic heparin-binding motive peptide, C², can have the sequence -(WZZWZW)_{M} (SEQ ID NO: 6), wherein independently and for each occurrence, W is A or G; Z is K or R; and each M is an integer independently selected from the group consisting of 2, 3, 4, 5, 6, 7, 8, 9, and 10.

The cationic heparin-binding motive peptide, C², can be -(AKKARA)_{M} (SEQ ID NO: 7). Each of the SAMs can include a hydrophobic portion (e.g., a C₁₆ hydrocarbon group), a beta-sheet forming segment (e.g., SEQ ID NO: 4, V₄K₄), and a cationic heparin-binding functional group (e.g., SEQ ID NO: 8, (AKKARA)₂). For example, a SAM can have the sequence C₁₆—V₄K₄G(AKKARA)₂-Q (SEQ ID NO: 9), wherein Q is not present, is —H, is —N(C═O)OH, or is —COOH. The beta-sheet forming segment can be —V₃K₃— (SEQ ID NO: 10). Table 1 summarizes sequences discussed above.

TABLE 1
Sequence Summary

| Sequence: | Summary: |
|---|---|
| SEQ ID | FA—A1—A2—A3—A4—A5—A6—A7—A8—A9—[A10—A11— |
| NO: 1 | A12—A13—A14—A15]n—Q |
| SEQ ID | FA—V—V—V—V—K—K—K—K—G—[A—K—K—A—R]n—Q |
| NO: 2 | |
| SEQ ID | —(X_{N})—(Z_{N})—B³ |
| NO: 3 | |
| SEQ ID | V₄K₄ |
| NO: 4 | |
| SEQ ID | V₄K₄G |
| NO: 5 | |
| SEQ ID | —(WZZWZW)_{M} |
| NO: 6 | |
| SEQ ID | —(AKKARA)_{M} |
| NO: 7 | |
| SEQ ID | (AKKARA)₂ |
| NO: 8 | |
| SEQ ID | C₁₆—V₄K₄G(AKKARA)₂—Q |
| NO: 9 | |
| SEQ ID | —V₃K₃— |
| NO: 10 | |
| SEQ ID | C₁₅—(C═O)—V₄K₄G(AKKAR)₂—Q |
| NO: 11 | |

FIG. 7 shows an example of a self-assembling molecule, a SAM, or a self-assembling biodegradable monomer (SABP) for a matrix. In FIG. 7, the cationic heparin-binding cardin-moiety can assemble with heparin before release from a 3D printed nanostructure. Alternatively, assembly with heparin can occur after release from a 3D printed nanostructure. Heparin can be exposed in the subject by trauma or surgery. During printing of a 3D printed nanostructure, heparin optionally can be included with the self-assembled nanostructure to form the SABP. In other examples, the SABP can be formed with other associated structures, along with a therapeutic agent, during, before, or after printing of a 3D printed nanostructure. The SABP can be released from a 3D printed nanostructure and subsequently associate with a biomolecule, target, or structure in a subject.

In the example of heparin, the mechanism of interaction between the cationic heparin-binding cardin-moiety can depend on interactions of positively charged amino acids (the cardin-moiety) with negatively charged heparan sulfate (HS) glycol self-assembled nanostructure in oglycan chains.

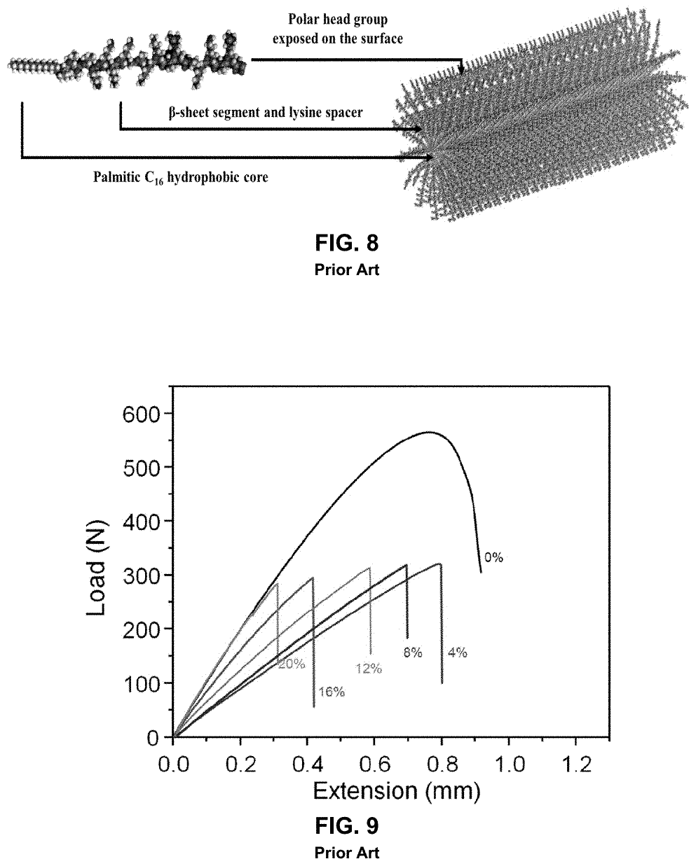

In the example shown in FIG. 8, a hydrophobic core can be utilized to encapsulate (or associate with) a therapeutic agent. The entire structure shown at the right of FIG. 8 can be a SABP, which is embedded in, is entrapped by, or is associated with a 3D printed structure. The 3D printed structure can release the SABP, along with therapeutics, upon exposure to NIR. The cylindrical structure shown at the right of FIG. 8 can have heparin bound or associated at the surface. Heparin can come from exposure of the SABP or self-assembled nanostructure to heparin after the SABP or self-assembled nanostructure is released from the 3D printed structure or the on-demand drug release system. Heparin can be added during printing of the 3D printed structure with the self-assembled nanostructure (and with a drug) to form the SABP during printing of the 3D printed structure (on-demand drug release system). Heparin can be added before implantation of an on-demand drug release system which was made utilizing the self-assembled nanostructure, 3D nanocomposite ink, and a drug. The self-assembled nanostructure can be synthesized and stored in wet or dry form before it is needed during 3D printing. The self-assembled nanostructure can take the form of the SABP (e.g., FIG. 8, right), after release from the 3D structure in the body, or before release, depending on the design of the drug-delivery system.

An ink for 3D printing can include the SAM. The ink can include a shape memory polymer and graphene, as the example shown in FIG. 1A. As discussed above, in FIG. 1A, BDE is bisphenol A diglycidyl ether; DA is decylamine, and PBE is poly(propylene glycol) bis(2-aminopropyl) ether. Graphene can be added from 0% to about 80% by weight. Optionally, graphene can be added from 0% to about 20% by weight. The SAM can form a structure, for example, as depicted in FIG. 8. The SAM can be associated with a therapeutic agent before, during, or after the 3D printing of the ink.

In the above-described methods, a therapeutic agent can be associated with or encapsulated by the SAM during the fabrication of a 3D printed structure, before, or after fabrication. The therapeutic agent encapsulated by the SAM in the form of a SABP. The SABP can include a binding moiety operative to target an area of the subject. The binding moiety can be a heparin binding moiety. The heparin binding moiety can, for example, target painful, stressed, or injured areas of the subject's body.

The 3D printed structure can be in the form of a medical device, such as a bone screw, a hip implant, a knee replacement, a shoulder implant, finger joint replacement, fixation for a ligament graft, a bone graft, an arthritic implant, a dental implant, a wrist implant, or a foot implant. The 3D printed structure can include a shape corresponding to a nerve, a myocardiocyte, or a sinoatrial node. The structure can be in the form of an implant deposited in the body during surgery for the purpose of releasing a therapeutic agent following the surgery.

A method of making a drug-delivery system is provided by the present technology. The method can include providing a source of EM. The method can further include implanting the 3D printed structure (drug-delivery platform) in a subject and can include the apparatus suitable for directing a signal towards the 3D printed structure, such that the drug-delivery platform is integrated into a subject and operable to deliver a therapeutic agent. The signal apparatus can be suitable for directing a NIR signal. While other bandwidths of EM can be utilized, IR radiation is utilized herein as an example of demonstrating the present technology.

In some examples, a kit for drug-delivery is provided. The kit includes a drug delivery platform including the 3D printed structure described above and, optionally, an apparatus suitable for directing a NIR signal to the implanted structure. The kit can include a plurality of 3D printed structures in a range of sizes, the sizes being appropriate for different subjects or different anatomical needs. The drug delivery structures of the kit can optionally contain one or more therapeutic agents, either different or the self-assembled nanostructuree for each structure. The structures of the kit can optionally be incorporated into, such as in the form of a coating or structural component, of a medical device designed for implantation into a subject.

The 3D printing techniques described herein can be utilized to form an initial shape of a 3D structure (or nanostructure) or drug-delivery system. The initial shape can then be formed to a second shape by heating with application of an external force. The second shape can be utilized for holding a therapeutic agent and for implantation into a subject. After exposure to NIR, the second shape can partially or substantially return to the initial shape, thereby releasing at least a portion of the therapeutic agent.

A therapeutic agent, for example, a drug, protein, cytokine, or growth factor used in the present technology, can be encapsulated or formulated for immediate or slow release. A 3D printing process can be utilized to form a coating of all or part of an implantable device, or any desired component or portion of such a device. The 3D printing process can be utilized to print an organ structure (such as bone, cartilage, vascular, heart, brain, spinal cord, etc.) or scaffold for cell attachment to form part or all of such an organ structure. The therapeutic agent can be embedded in a delivery matrix which can be designed to target specific cells, tissues, or organs or designed to diagnose and/or treat a disease or medical condition, such as pain, cancer, osteoporosis, or Alzheimer's disease, for example.

Examples of suitable therapeutic agents include analgesics, such as COX-2 inhibitors, analgesic combinations (including the narcotic or opioid analgesic combinations), antimigraine agents, salicylates, nonsteroidal anti-inflammatory drugs (NSAIDs), paracetamol, voltage-gated Na channel blockers, and multimodal agents.

A biodegradable polymer or a SABP can be designed to target a specific location within a subject. The SABP can include SAMs or self-assembling nanostructures having a designed targeting moiety. For example, a cationic heparin-binding self-assembled nanostructure is shown in the example of FIG. 7. In FIG. 7, the variable Z can be selected from the group consisting of 1, 2, 3, 4, 5, 6, 7, 8, 9, 10, 11, 12, 13, 14, and 15. The heparin targeting moiety can be directed to areas of pain or damage within a subject to deliver an analgesic payload. Depending on the self-assembled nanostructure and the drug, a therapeutic agent can be encapsulated in or associated with the SABP. FIG. 8 illustrates a self-assembled molecular delivery system or SABP that can associate with a therapeutic agent as a delivery matrix. For example, a hydrophobic drug can be encapsulated at the hydrophobic center of the structure shown at the right of FIG. 8.

Heparin is known to be stored within the secretory granules of mast cells and released into vasculature at sites of tissue injury. The polar head groups shown at the right of the structure in FIG. 8 can include positive charges that associate with heparin. The heparin targeting self-assembled nanostructure shown in FIG. 7 can be incorporated in a 3D printed structure (or on-demand drug release system) during fabrication, for example, during 3D printing or after.

The on-demand drug release system can be formed by 3D printing which forms a 3D structure or nanostructure. The structure can be a “4D structure” that can change form over time in response to an environmental stimulus or due to a planned form change, such as in response to NIR or other electromagnetic radiation.

To form an exemplary on-demand drug release system using a 3D printed structure, a smart epoxy with a shape memory property can be synthesized and doped with varying amounts of graphene (see FIG. 1A, FIG. 1D, and FIG. 9). The graphene-doped structures exhibit an excellent photothermal effect. Compared to other 3D printed materials, the NIR responsive 3D printed composite possess a dynamically and remotely controllable spatiotemporal transformation. For example, a proof-of-concept 3D printed brain-shaped construct is shown in FIG. 6B. The brain construct can incorporate neural stem cell growth. By the combination of its electroconductive and optoelectronic properties, the 3D neural cell-laden construct exhibits excellent neural stem cell growth and differentiation. The brain construct, or other organ constructs or scaffolds, can be designed to deliver drugs or therapeutics, over time, by embedding SABP as shown in FIG. 8 along with an associated therapeutic agent. The brain construct can respond to external stimuli, such as NIR (FIG. 4A), and the response can release the SABP with associated drug.

The graphene in the 3D structure and the self-assembled nanostructure can combine via a chemical attraction. The therapeutic agent can be incorporated in or with the self-assembled nanostructure, with the self-assembled nanostructure assembled around the drug based on surface energy attraction. After implantation, the NIR can cause the graphene to increase in temperature, disrupting the self-assembled bonds (self-assembled nanostructure bonds) to release the drug or a therapeutic inside or associated with the SABP. The SABP can self-assemble when added to the 3D nanocomposite ink during printing, because the ink allows for hydrogen and secondary bonds to form. The drug can interact with the self-assembled nanostructure/SABP (and the self-assembled nanostructure/SABP can interact with the 3D structure) through hydrogen bonds, charge, or charge (electrostatics), π-π interactions, van der Waals forces, or can be associated with, sufficient to only release the therapy on-demand.

The 3D printed structures can incorporate nanostructures such as nanoparticles, nanofilms, nanorods, nanostars, nanotubes, nanoplatelets, etc., and/or graphene as an electrically active component to electrically stimulate cells such as neurons, cardiovascular cells, osteoblasts, or chondrocytes. The above described constructs (e.g., FIG. 4A) can be used to regenerate various organs (such as the cartilage, vascular, heart, brain, spinal cord) coupled with simultaneous pain medication therapeutics such as the new highly specialized opioid and non-opioid analgesics. The constructs illustrated above can be used for signal transmission of neural cells, essentially contributing to improved therapeutics in neurological diseases such as Alzheimer's and Parkinson's disease (and potential rare diseases). The use of 3D printed constructs can function as or in conjunction with a pacemaker, or contributing to the autonomous beating of cardiomyocytes coupled with electroactive tissue regeneration to prevent myocardial damage or further damage.

The above constructs can be utilized to develop circuitries coupled with artificial intelligence for improving electroactivity of engineered tissue without need for an external stimulation device. Given their electrical conductivity and possible nanometer scale or micrometer scale, the SABPs also can be used in a brain or neural interface with other tissue or with an artificial device, either implanted or outside the body.

The 3D printed structures of the present technology are well-suited for forming orthopedic implants that can provide on-demand analgesia (compatible with newly developed non-opioid analgesics) optionally together with antibiotics, anti-clotting agents, anti-inflammatory agents, or other therapeutic agents.

An example method of making an implantable 3D structure holding a therapeutic agent can include the steps of: a) providing a SMP, a therapeutic agent, and optionally graphene and a biodegradable polymer; b) printing or forming the SMP with the optional graphene into a permanent shape; c) dispersing the therapeutic agent and the biodegradable polymer on or near the permanent shape; d) heating the permanent shape above a Tg of the SMP and applying an external force to the permanent shape, whereby a temporary shape is formed at least partially around the therapeutic agent; e) cooling the temporary shape below the Tg, whereby the temporary shape provides the implantable 3D structure. The therapeutic agent can be associated with a biodegradable polymer at any step. The therapeutic agent can be physically entrapped by the implantable 3D structure, in pores or lamellar microstructures, associated with the biodegradable polymer in a matrix with the 3D structure, or a combination thereof.

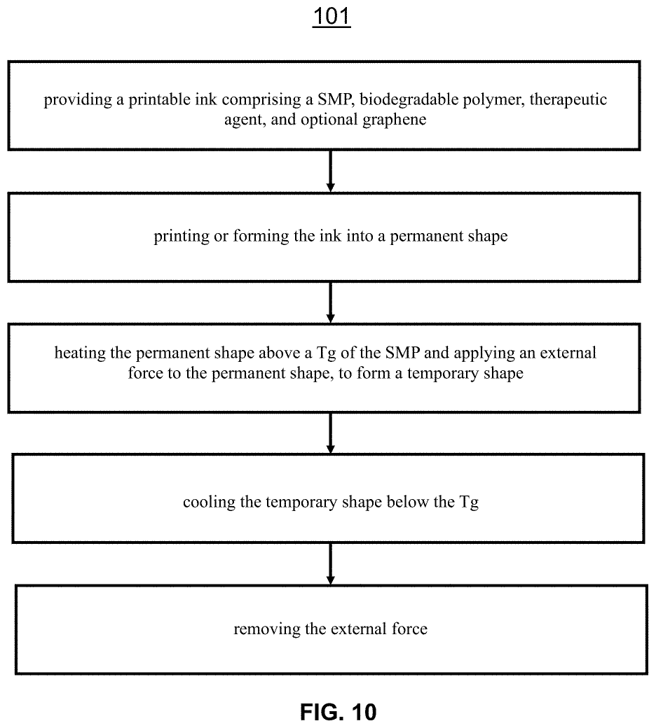

FIG. 10 shows an example flow diagram 101 for making an implantable 3D structure holding a therapeutic agent. The method can include the steps of: providing a printable ink comprising a SMP, a biodegradable polymer, a therapeutic agent, and optionally graphene; printing or forming the ink into a permanent shape; heating the permanent shape above a Tg of the SMP and applying an external force to the permanent shape, whereby a temporary shape is formed; applying the external force while cooling the temporary shape below the Tg, then removing the external force; whereby the temporary shape provides the implantable 3D structure. The biodegradable polymer and the therapeutic agent can be associated with the temporary shape, in pores or lamellar microstructures (e.g., FIGS. 2E-2I) of the temporary shape, held by the temporary shape, or a combination thereof.

The 3D printing techniques herein can be utilized to form an initial shape of a 3D structure or drug-delivery system. The initial shape can include the self-assembled nanostructure, SABPs, drug, or any combination thereof. Alternatively, the initial shape can be printed without including the self-assembled nanostructure, SABPs, or drug. Then, the initial shape can be formed to a second shape by application of an external force. The second shape can be stable and firm at body temperature. After forming the second shape, the self-assembled nanostructure, and drug can be added to the second shape, for example, in selected areas or in pre-designed release areas. Then, the second shape can be utilized for implantation into a subject. After exposure to NIR (within the subject), the second shape can partially or mostly return to the initial shape, the return to the initial shape such that it releases the self-assembled nanostructure (or SABPs) and drug. The self-assembled nanostructure can form into the SABPs before release or after release into the body. For example, if heparin is added to the self-assembled nanostructure during formation of the 3D structure (drug-delivery system), the self-assembled nanostructure can form SABPs before release into the body.

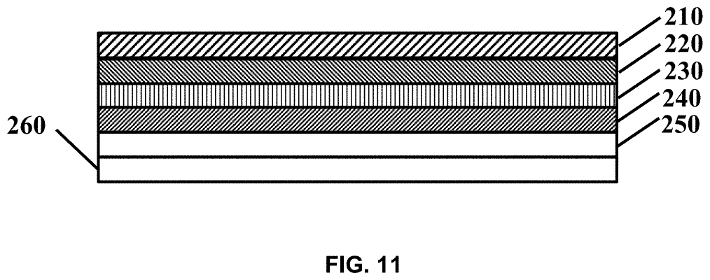

The drug-delivery technology can be secured. Light discussed herein can be unpolarized or polarized in a linear, circular, or elliptical polarization. The receiving agent capable of absorbing a bandwidth of EM can be configured to only respond to (to transduce) light including or consisting of a specific polarization or a specific characterization (e.g., timed pulses). For example, the thermally absorbing agent capable of absorbing NIR radiation can include one or more polarization 2D layers (e.g., filters) so that only NIR radiation with a specific polarization reaches or is absorbed by the thermally absorbing agent. The polarization filter(s) can be configured to only transmit circularly polarized light of a left-handed (counter-clockwise viewed by receiver) or right-handed (clockwise circularly polarized if viewed by the receiver) circular polarization, for example, by combining a quarter-wave plate with a linearly polarized filter. Circular polarization filters can be provided in a thin film or 2D layer over the thermally absorbing agent or over the 3D structure. FIG. 11 shows a cross-sectional view of example layers of an implantable 3D structure holding and including a therapeutic agent. Layer 210 is an optional EM filter layer over a receiving agent layer 220. Layer 230 includes the SMP. Optional layer 240 is a matrix including or consisting of a biodegradable polymer. Optional layer 250 is an encapsulating formulation or encapsulating polymer over the therapeutic agent 260. The receiving agent 220, the layer 230 including the SMP, and the therapeutic agent 260 are required. In this example, the NIR wand or NIR emitter accessible only by a health care provider can include a photoelastic modulator (PEM), a polarimeter, or both, to ensure that only the health care provider has the capability for providing the specific NIR radiation to cause a release of therapeutic agent.

In another example, the thermally absorbing agent can include or consist of chiral bond including agents, polymers, bonds, or chiral molecules configured to preferentially absorb left-handed circularly polarized light over right-handed circularly polarized light falling in a specific bandwidth, or vice versa right-handed over left-handed. Circular dichroism (CD) is a dichroism involving circularly polarized light (i.e., the differential absorption of left- and right-handed circularly polarized light) and can be referred to as specific forms of dichroism (e.g., vibrational circular dichroism) depending on the bandwidth of the absorbed light. A NIR wand or a NIR emitter accessible only by a health care provider can provide the specific polarization required for absorption by the thermally absorbing agent. The use of linear or circularly polarized light can ensure that only a health care provider can provide NIR radiation with the specific polarization to cause a release of the therapeutic agent. The example of a PEM tuned to specific wavelengths with right/left-handed polarization can provide for security up to the degree that the NIR wand or NIR emitter can be specific for a therapeutic release in one subject and not capable of providing therapeutic release for another subject.

In another example, light with one wavelength/polarization can be utilized to unlock a portion of the outer 3D structure, while light with a different wavelength/polarization can be utilized to cause the release of the therapeutic agent by moving an inner or a different portion of the outer 3D structure.

The on-demand implantable drug release system can take almost any shape or form. A 4D printing process as described herein can be utilized to form almost any coating, shape, or portion of an implantable device that can dynamically transform its form or nanostructure in response to a remote stimulus, with the transformation of form or nanostructure pre-determined, for example by glass transition temperature, during design and the fabrication process. Implanted devices of the present technology, which are activated by NIR, can be coupled with the use of an external device that provides the NIR. An external device can be, for example, in the form of a watch or wand used to release and/or collect molecules within the body, such as pain killers or antibiotics, on-demand to enhance therapeutic drug monitoring or reversal of overdose.

### EXAMPLES

### Example 1: Preparation of 3D Printing Ink and Printability

Bisphenol A diglycidyl ether, decylamine and poly(propylene glycol) bis(2-aminopropyl) ether were purchased from Sigma. By varying the formulations of these components, different epoxy polymers were synthesized. Three monomer chemicals were melted and mixed in an oven at 50° C. for 5 minutes. After thermally curing at 70° C. for 48 hours, the shape memory properties of epoxy self-assembled nanostructures were investigated to obtain an optimal formulation. Graphene nanoplatelets (6-8 nm thick×5 μm wide) were obtained from Strem Chemicals Inc. The graphene nanoplatelets with different concentrations were weighed and added in the monomer mixture. The nanocomposite inks were melted at 50° C. for 5 minutes, uniformly dispersed by mechanically stirring for 5 minutes, and then sonicated for 10 minutes. The viscosity of the inks was analyzed with an MCR 302 rheometer (Anton Paar, see FIG. 1B), and the inks were placed on cone plates of 25 mm diameter and a gap of 104 μm.

### Example 2: Structure Design and Printing

A dual printing technique was developed in this experiment, which included fused deposition modeling and extrusion printing. The structures were designed with the software Autodesk12 (Autodesk Inc.) and saved as .stl files. After the .stl files were uploaded, the pre-molds were manufactured by fused deposition modeling printer with polyvinyl alcohol (PVA) filament (MakerBot Industries) similar to previous papers [36, 37]. The typical parameters including infill density (100%), the printing speed (25 mm/s), printing temperature (200° C.) and layer height (200 μm) were used. Other parameters assigned in Slic3r include: 150 μm first layer height; vertical shells—perimeters 0; horizontal shells—solid layers, top 0, bottom 0; 90° infill angle, 10 mm² solid infill threshold area; skirt, loop 0; extrusion width, the first layer 0%. The nanocomposite inks were preheated to 50° C. and extruded into printed molds at room temperature using a customized extrusion printer developed for the specific purpose in the lab. The printing parameters were set to 100% infill, the printing speed of 10 mm/s, and 0.5-1 mm in layer height. The 3D constructs were cured at room temperature for 24 hours followed by post-curing at 70° C. in an oven for 24 hours. After polymerization, the self-assembled nanostructures were washed overnight in ethanol to remove unpolymerized ink and then soaked in boiled water for several times to purify the self-assembled nanostructures. The Raman spectra of graphene and nanocomposites were conducted with 3,000-100 cm⁻¹ wavelength range using a LabRAM HR Evolution Raman spectroscope (HORIBA Scientific).

### Example 3: Microstructural Morphologies, Thermal Properties, and Mechanical Properties

Morphological analysis of the cured self-assembled nanostructures was performed after they were gold sputter-coating via an extreme high-resolution field emission scanning electron microscopy (SEM) mode (FEI FIBSEM) under an accelerating voltage of 5 kV. The Tg of the self-assembled nanostructures was measured with a multi-cell differential scanning calorimeter (MC DSC) from TA Instruments (New Castle, Del.) at a programmed ramp rate of 1° C./min. The self-assembled nanostructureple was first heated from 25 to 150° C., and held at 150° C. for 1 minute. Then the self-assembled nanostructureple was cooled from 150 to −30° C., and held at −30° C. for 1 minute. A second cycle was conducted: heating from −30 to 150° C., holding 1 minute and decreasing from 150 to −30° C. The second cycle was used to determine the Tg. Tensile testing of the self-assembled nanostructures was conducted using a uniaxial mechanical tester (MTS Criterion Model 43). The self-assembled nanostructures were mounted on the 5 kN load cell and pulled at a rate of 0.5 mm/min until failure. Data were taken using TW software and Young's modulus was determined by the linear portion of the tensile stress-strain curve.

### Example 4: Shape Memory Properties and Photothermal Properties

Shape memory tests were conducted according to a previously reported method [18]. The self-assembled nanostructures were printed into 50 mm×5 mm strips, and folded 180° at 60° C. into a “U” shape with an inner radius of 10 mm, and kept at this temperature for 10 minutes. The self-assembled nanostructures were then immediately cooled to room temperature and maintained at this temperature for an additional 10 minutes. The fixed angle of the specimen was determined and recorded as θ_{fixed} The strips were then immersed in different preset temperature to recover the permanent shape. The final angle of the specimen was determined and recorded as θ_{final}. Shape fixity (Rf) and shape recovery (Rr) were calculated by the following equations: Rf=θ_{fixed}/180×100%, and Rr=(θ_{fixed}−θ_{final})/θ_{fixed}×100%.

After testing the thermally-triggered shape change, its photothermal material's properties were further investigated. A customized NIR laser device composed of PSU-III-LED power (0-2,000 mW, 808 nm) and 400 μm fiber cable was utilized for studying the shape recovery process. The relationship between NIR illumination and temperature change was systemically analyzed by varying exposure time, laser energy, and distance. The dynamic shape change of different models was recorded with a PowerShot ELPH 360HS Cannon camera. The temperature data and thermal images were collected using Visual IR Thermometer (FLUKE).

### Example 5: Electrical and Optoelectronic Properties

Cyclic voltammetry (CV) and Amperometric i-t curves of the self-assembled nanostructures were conducted on a DY2000 Series Multi-channel Potentiostat (Digi-Ivy, Inc.) using Ag/AgCl and Pt as the reference and counter electrodes, respectively. The self-assembled nanostructure was used as the working electrode, and the scan rate was 50 mV/s. For the optoelectronic study, the NIR laser was set to 2,000 mW, and the initial potential on the working electrode was 0 V. The electrical conductivity of self-assembled nanostructures was measured using 4-Point sheet resistance meter (R-CHEK).

### Example 6: Cell Culture and Differentiation

NSCs cloned from mouse neuroectoderm (NE-4C) were purchased from American Type Culture Collection (ATCC). NSCs were cultured in Eagle's minimum essential medium (ATCC) supplemented with 5% (v/v) fetal bovine serum and 1% (v/v) L-glutamine. For neuronal differentiation studies of NSCs, cells were cultured in the aforementioned complete medium supplemented with all-trans retinoic acid (RA, 10⁻⁶ M) [35]. All cells were incubated in a 95% humidified atmosphere with 5% CO2 at 37° C.

### Example 7: Cell Viability, Proliferation, and Morphology

NSCs were seeded on the constructs at a density of 5×10⁴ cells/mL and continuously cultured for 1, 3, and 7 days. At the predetermined time interval, the culture medium containing 10% CCK-8 solution (Dojindo, Japan) was added and incubated for 2 hours. 200 μL of medium was transferred into a 96-well plate, and the absorbance at a wavelength of 450 nm was quantified by a spectrophotometer (Thermo, USA). Specifically, at each predetermined time, all constructs were fixed with 10% formalin for 15 minutes and then permeabilized with 0.2% Triton-100 for 10 minutes. The self-assembled nanostructures were then stained with a Texas Red-X phalloidin solution (1:100) for 30 minutes, followed by 4′, 6-diamidino-2-phenylindole (DAPI) (1:1,000) solution for another 5 minutes. The NSC morphology on the constructs was observed using laser confocal microscopy (Carl Zeiss LSM 710). Additionally, GFP-NSCs (NE-GFP-4C, ATCC) were seeded on the constructs at a density of 5×10⁴ cells/mL and cultured for 24 hours. By varying temperature in relation to laser exposure, cell viability was measured using CCK-8 kit and observed using confocal microscopy.

### Example 8: Immunofluorescence Staining

NSCs were seeded at a density of 3×10⁴ cells/mL on each self-assembled nanostructure and incubated in the aforementioned differentiation medium for 14 days to evaluate the neuronal differentiation. The self-assembled nanostructures were fixed with 10% formalin for 15 minutes followed by permeabilization in 0.2% Triton100 solution for 10 minutes at room temperature. Then the self-assembled nanostructures were incubated with blocking solution (containing 1% bovine serum albumin (BSA), 0.1% Tween 20 and 0.3 M glycine in PBS) for 2 hours. The first primary antibody of mouse anti-TuJ1 (1:1,000), rabbit anti-GFAP antibody (1:500) and rabbit anti-MAP2 antibody (1:500) were gently mixed with self-assembled nanostructures overnight at 4° C. Next, the secondary antibodies of goat anti-mouse Alexa Fluor 594 (1:1,000) and goat anti-rabbit Alexa Fluor 488 (1:1,000) were incubated with self-assembled nanostructures in the dark for 2 hours at room temperature, followed by DAPI (1:1,000) solution incubation for 5 minutes. The immunofluorescence images were taken using confocal microscopy.

### REFERENCES

Scholl L., Seth P., Kariisa M., Wilson N., Baldwin G. Drug and Opioid-Involved Overdose Deaths—United States, 2013-2017. MMWR Morb Mortal Wkly Rep. 2018; 67(5152):1419-1427. Published 2018 Jan. 4. doi:10.15585/mmwr.mm675152e1
World Drug Report 2019, United Nations Office on Drugs and Crime (UNODC), Booklet 3 “Depressants”, pg. 23, (wdr.unodc.org/wdr2019/).
Miao, S. D.; Castro, N.; Nowicki, M.; Xia, L.; Cui, H. T.; Zhou, X.; Zhu, W.; Lee, S. J.; Sarkar, K.; Vozzi, G. et al. 3D printing of polymeric materials for tissue and organ regeneration. Mater. Today 2017, 20, 577-591.
Shin, D. G.; Kim, T. H.; Kim, D. E. Review of 3D printing materials and their properties. Int. J. Precis. Eng. Manuf.-Green Tech. 2017, 4, 349-357.
Cui, H. T.; Nowicki, M.; Fisher, J. P.; Zhang, L. G. bioprinting for organ regeneration. Adv. Healthc. Mater. 2017, 6, 1601118.
Cui, H. T.; Miao, S. D.; Esworthy, T.; Zhou, X.; Lee, S. J.; Liu, C. Y.; Yu, Z. X.; Fisher, J. P.; Mohiuddin, M.; Zhang, L. G. bioprinting for cardiovascular regeneration and pharmacology. Adv. Drug Deliv. Rev. 2018, 132, 252-269.
Deng, C. J.; Yao, Q. Q.; Feng, C.; Li, J. Y.; Wang, L. M.; Cheng, G. F.; Shi, M. C.; Chen, L.; Chang, J.; Wu, C. T. printing of bilineage constructive biomaterials for bone and cartilage regeneration. Adv. Funct. Mater. 2017, 27, 1703117.
Yang, B. W.; Yin, J. H.; Chen, Y.; Pan, S. S.; Yao, H. L.; Gao, Y. S.; Shi, J. L. 2D-black-phosphorus-reinforced-printed scaffolds: A stepwise countermeasure for osteosarcoma. Adv. Mater. 2018, 30, 1705611.
Lalwani, G.; D'Agati, M.; Khan, A. M.; Sitharaman, B. Toxicology of graphene-based nanomaterials. Adv. Drug Deliv. Rev. 2016, 105, 109-144.
Zhao, Q. L.; Wang, J.; Cui, H. Q.; Chen, H. X.; Wang, Y. L.; Du, X. M. Programmed shape-morphing scaffolds enabling facile endothelialization. Adv. Funct. Mater. 2018, 28, 1801027.
Li, Y. C.; Zhang, Y. S.; Akpek, A.; Shin, S. R.; Khademhosseini, A. 3D bioprinting: The next-generation technology for biofabrication enabled by stimuli-responsive materials. Biofabrication 2016, 9, 012001.
Choi, J.; Kwon, O. C.; Jo, W.; Lee, H. J.; Moon, M. W. 3D printing technology: A review. Print. Addit. Manuf. 2015, 2, 159-167.
Miao, S. D.; Cui, H. T.; Nowicki, M.; Xia, L.; Zhou, X.; Lee, S. J.; Zhu, W.; Sarkar, K.; Zhang, Z. Y.; Zhang, L. G. Stereolithographic 3D bioprinting of multiresponsive structures for neural engineering. Adv. Biosyst. 2018, 2, 1800101.
Gladman, A. S.; Matsumoto, E. A.; Nuzzo, R. G.; Mahadevan, L.; Lewis, J. A. Biomimetic 3D printing. Nat. Mater. 2016, 15, 413-418.
Huang, L. M.; Jiang, R. Q.; Wu, J. J.; Song, J. Z.; Bai, H.; Li, B. G.; Zhao, Q.; Xie, T. Ultrafast digital printing toward 3D shape changing materials. Adv. Mater. 2017, 29, 1605390.
Lee, A. Y.; An, J.; Chua, C. K. Two-way 3D printing: A review on the reversibility of-printed shape memory materials. Engineering 2017, 3, 663-674.
Ding, Z.; Yuan, C.; Peng, X. R.; Wang, T. J.; Qi, H. J.; Dunn, M. L. Direct 3D printing via active composite materials. Sci. Adv. 2017, 3, e1602890.
Li, W. B.; Liu, Y. J.; Leng, J. S. Selectively actuated multi-shape memory effect of a polymer multicomposite. J. Mater. Chem. A 2015, 3, 24532-24539.
Wei, H. Q.; Zhang, Q. W.; Yao, Y. T.; Liu, L. W.; Liu, Y. J.; Leng, J. S. Direct-write fabrication of 3D active shape-changing structures based on a shape memory polymer and its nanocomposite. ACS Appl. Mater. Interfaces 2017, 9, 876-883.
Miao, S. D.; Zhu, W.; Castro, N. J.; Nowicki, M.; Zhou, X.; Cui, H. T.; Fisher, J. P.; Zhang, L. G. 3D printing smart biomedical scaffolds with soybean oil epoxidized acrylate. Sci. Rep. 2016, 6, 27226.
Miao, S. D.; Cui, H. T.; Nowicki, M.; Lee, S. J.; Almeida, J.; Zhou, X.; Zhu, W.; Yao, X. L.; Masood, F.; Plesniak, M. W. et al. Photolithographic-stereolithographic-tandem fabrication of 3D smart scaffolds for improved stem cell cardiomyogenic differentiation. Biofabrication 2018, 10, 035007.
Miao, S. D.; Zhu, W.; Castro, N. J.; Leng, J. S.; Zhang, L. G. Four-dimensional printing hierarchy scaffolds with highly biocompatible smart polymers for tissue engineering applications. Tissue Eng. Part C Methods 2016, 22, 952-963.
Chen, A. C.; Chatterjee, S. Nanomaterials based electrochemical sensors for biomedical applications. Chem. Soc. Rev. 2013, 42, 5425-5438.
Chung, C.; Kim, Y. K.; Shin, D.; Ryoo, S. R.; Hong, B. H.; Min, D. H. Biomedical applications of graphene and graphene oxide. Acc. Chem. Res. 2013, 46, 2211-2224.
Gao, J. H.; Gu, H. W.; Xu, B. Multifunctional magnetic nanoparticles: Design, synthesis, and biomedical applications. Acc. Chem. Res. 2009, 42, 1097-1107.
Blum, A. P.; Kammeyer, J. K.; Rush, A. M.; Callmann, C. E.; Hahn, M. E.; Gianneschi, N. C. Stimuli-responsive nanomaterials for biomedical applications. J. Am. Chem. Soc. 2015, 137, 2140-2154.
Shen, H.; Zhang, L. M.; Liu, M.; Zhang, Z. J. Biomedical applications of graphene. Theranostics 2012, 2, 283-294.
Yang, Y. Q.; Asiri, A. M.; Tang, Z. W.; Du, D.; Lin, Y. H. Graphene based materials for biomedical applications. Mater. Today 2013, 16, 365-373.
Jakus, A. E.; Secor, E. B.; Rutz, A. L.; Jordan, S. W.; Herself-assembled nanostructure, M. C.; Shah,
R. N. Three-dimensional printing of high-content graphene scaffolds for electronic and biomedical applications. ACS Nano 2015, 9, 4636-4648.
Zhou, X.; Nowicki, M.; Cui, H. T.; Zhu, W.; Fang, X. Q.; Miao, S. D.; Lee,
S. J.; Keidar, M.; Zhang, L. J. G. bioprinted graphene oxide-incorporated matrix for promoting chondrogenic differentiation of human bone marrow mesenchymal stem cells. Carbon 2017, 116, 615-624.
Xie, T.; Rousseau, I. A. Facile tailoring of thermal transition temperatures of epoxy shape memory polymers. Polymer 2009, 50, 1852-1856.
Wei, J. C.; Atif, R.; Vo, T.; Inam, F. Graphene nanoplatelets in epoxy system: Dispersion, reaggregation, and mechanical properties of nanocomposites. J. Nanomater. 2015, 2015, Article ID 561742.
Cui, H. T.; Liu, Y. D.; Cheng, Y. L.; Zhang, Z.; Zhang, P. B.; Chen, X. S.; Wei, Y. In vitro study of electroactive tetraaniline-containing thermosensitive hydrogels for cardiac tissue engineering. Biomacromolecules 2014, 15, 1115-1123.
Zhu, W.; Ye, T.; Lee, S. J.; Cui, H. T.; Miao, S. D.; Zhou, X.; Shuai, D. M.; Zhang, L. G. Enhanced neural stem cell functions in conductive annealed carbon nanofibrous scaffolds with electrical stimulation. Nanomedicine 2018, 14, 2485-2494.
Cui, H. T.; Wang, Y.; Cui, L. G.; Zhang, P. B.; Wang, X. H.; Wei, Y.; Chen, X. S. In vitro studies on regulation of osteogenic activities by electrical stimulus on biodegradable electroactive polyelectrolyte multilayers. Biomacromolecules 2014, 15, 3146-3157.
Cui, H. T.; Shao, J.; Wang, Y.; Zhang, P. B.; Chen, X. S.; Wei, Y. PLA-PEG-PLA and its electroactive tetraaniline copolymer as multi-interactive injectable hydrogels for tissue engineering. Biomacromolecules 2013, 14, 1904-1912.
Zhou, X.; Cui, H. T.; Nowicki, M.; Miao, S. D.; Lee, S. J.; Masood, F.; Harris, B. T.; Zhang, L. G. Three-dimensional-bioprinted dopamine-based matrix for promoting neural regeneration. ACS Appl. Mater. Interfaces 2018, 10, 8993-9001.
Cui, H. T.; Zhu, W.; Holmes, B.; Zhang, L. G. Biologically inspired smart release system based on bioprinted perfused scaffold for vascularized tissue regeneration. Adv. Sci. 2016, 3, 1600058.
Cui, H. T.; Zhu, W.; Nowicki, M.; Zhou, X.; Khademhosseini, A.; Zhang, L. G. Hierarchical fabrication of engineered vascularized bone biphasic constructs via dual bioprinting: Integrating regional bioactive factors into architectural design. Adv. Healthc. Mater. 2016, 5, 2174-2181.

The content of the ASCII text file of the sequence listing named Sequence-Listing-19815-0771_ST25, having a size of 4.68 kb and a creation date of 18 May 2022, and electronically submitted via EFS-Web on 18 May 2022, is incorporated herein by reference in its entirety.

1. An implantable drug release structure comprising:
a shape memory polymer having a glass transition temperature greater than about 37° C.;
a thermal transduction agent capable of absorbing near infrared light and inducing thermal motion in response thereto, wherein the thermal transduction agent is non-covalently associated with said shape memory polymer; and
a drug non-covalently associated with the implantable drug release structure below said glass transition temperature.

2. The implantable drug release structure of claim 1, wherein the drug is disposed within a polymer matrix comprising the shape memory polymer.

3. The implantable drug release structure of claim 2, wherein the drug is embedded within the polymer matrix.

4. The implantable drug release structure of claim 1, wherein the drug is disposed within a compartment formed by the drug release structure in a closed configuration, and wherein said compartment opens above the glass transition temperature to release the drug.

5. The implantable drug release structure of claim 1, wherein the drug is associated with a biodegradable polymer, said biodegradable polymer associated with the implantable drug release structure.

6. The implantable drug release structure of claim 5, wherein the biodegradable polymer comprises an amphiphilic peptide.

7. The implantable drug release structure of claim 1, wherein the drug is associated with nanoparticles, said nanoparticles associated with the implantable drug release structure.

8. The implantable drug release structure of claim 1, wherein the thermal transduction agent comprises graphene.

9. The implantable drug release structure of claim 1, wherein the thermal transduction agent is present in an amount up to about 20 weight % of the implantable drug release structure.

10. The implantable drug release structure of claim 1, wherein the shape memory polymer comprises an epoxy monomer, an aliphatic diamine crosslinker, and a crosslinking modulator.

11. The implantable drug release structure of claim 10, wherein the crosslinking modulator comprises decylamine.

12. The implantable drug release structure of claim 1, wherein the glass transition temperature is about 45° C.

13. The implantable drug release structure of claim 1, wherein the drug is an analgesic agent, an anti-inflammatory agent, an antibiotic, or a combination thereof.

14. The implantable drug release structure of claim 1, wherein the shape memory polymer changes form when heated to a temperature above the glass transition temperature, said change in form leading to release of the drug from the implantable drug release structure.

15. The implantable drug release structure of claim 1, wherein the thermal transduction agent is associated with the shape memory polymer via π-π interactions, cation-π interactions, ionic interactions, van der Waals interactions, hydrogen bonding interactions, or a combination thereof.

16. A method of administering a drug to a subject, the method comprising:
(a) providing an implantable drug release structure comprising:
a shape memory polymer having a glass transition temperature greater than about 37° C.;
a thermal transduction agent capable of absorbing near infrared light and inducing thermal motion in response thereto, wherein the thermal transduction agent is non-covalently associated with said shape memory polymer; and
a drug non-covalently associated with the implantable drug release structure below said glass transition temperature;
(b) implanting the implantable structure in the subject; and
(c) irradiating the subject with near infrared radiation in a body region comprising the implanted drug release structure, whereby the thermal transduction agent absorbs the near infrared radiation. thereby inducing a shape change of the implanted drug release structure and releasing the drug.

17. The method of claim 16, wherein step (c) is performed by trained medical personnel or in a doctor's office, hospital, or other medical facility.

18. The method of claim 16, further comprising, prior to step (b):
(b0) performing a surgical procedure at a site in the subject's body; and
wherein step (b) comprises implanting the implantable drug release structure at or near the surgical site.

19. The method of claim 16, wherein the implantable drug release structure is an implantable medical device or forms a portion of an implantable medical device.

20. The method of claim 19, wherein the implantable medical device is an orthopedic device and the surgical procedure is an orthopedic surgical procedure.

21. The method of claim 16, wherein the drug is an analgesic agent, an anti-inflammatory agent, an antibiotic, or any combination thereof.

22. A method of making an implantable drug release structure, the method comprising the steps of:
(a) providing an ink for 3D printing, the ink comprising a shape memory polymer, a thermal transduction agent, and a drug;
(b) printing the ink into a 3D object having a first shape;
(c) heating the object to above a glass transition temperature of the shape memory polymer;
(d) changing the heated object's shape from the first shape to a second shape; and
(e) cooling the object to below the glass transition temperature while maintaining the second shape.

23. A kit for preparing an implantable drug release structure, the kit comprising:
(i) an ink for 3D printing, the ink comprising a shape memory polymer, a thermal transduction agent; and
(ii) instructions for preparing an implantable drug release structure using the ink and the method of claim 22; and
(iii) optionally one or more drugs for addition to the ink and use in the method to prepare the implantable drug release structure; and
(iv) optionally one or more implantable medical devices for use as a substrate during said 3D printing.

24. An implantable medical device comprising one or more implantable drug release structures of claim 1 attached to the device or present as a coating or portion thereof of the device.


SEQUENCE LISTING (11 sequences)

SEQ ID NO: 1 - protein, 15 aa, Artificial Sequence
  Self-assembling monomer peptide
  Xaa (1)..(8): Xaa can be any naturally occurrng amino acid.
  MISC_FEATURE (1)..(8)
  Xaa (1)..(1): Amino terminus is acylated with a C2-C30 fatty acid.
  Xaa (9)..(9): Xaa can be absent or any naturally occurrng amino acid.
  Xaa (10)..(14): Xaa can be any naturally occurrng amino acid.
  Xaa (15)..(15): Xaa can be absent or can be glycine.
XXXXXXXXXXXXXXX

SEQ ID NO: 2 - protein, 14 aa, Artificial Sequence
  Self-assembling monomer peptide
  MOD_RES (1)..(1): Amino terminus is acylated with a C2-C30 fatty acid.
  MISC_FEATURE (10)..(14): Segment from residues 10-14 optionally can be repeated 1-14 times.
VVVVKKKKGAKKAR

SEQ ID NO: 3 - protein, 3 aa, Artificial Sequence
  Self-assembling monomer peptide
  MISC_FEATURE (1)..(1): Xaa is I, L, or V and optionally can be repeated 1-9 times.
  MISC_FEATURE (1)..(1): Amino terminus is acylated with a C2-C30 fatty acid.
  MISC_FEATURE (2)..(2): Xaa is K or R and optionally can be repeated 1-9 times.
  MISC_FEATURE (3)..(3): Xaa is A, G, or absent.
XXX

SEQ ID NO: 4 - protein, 8 aa, Artificial Sequence
  Self-assembling monomer peptide
  MISC_FEATURE (1)..(1): Amino terminus is acylated with a C2-C30 fatty acid.
VVVVKKKK

SEQ ID NO: 5 - protein, 9 aa, Artificial Sequence
  Self-assembling monomer peptide
  MISC_FEATURE (1)..(1): Amino terminus is acylated with a C2-C30 fatty acid.
VVVVKKKKG

SEQ ID NO: 6 - protein, 6 aa, Artificial Sequence
  Hepartin-binding peptide
  misc_feature (1)..(1): Xaa is A or G.
  misc_feature (1)..(1): Amino terminus is acylated with a C2-C30 fatty acid.
  misc_feature (2)..(3): Xaa is K or R.
  misc_feature (4)..(4): Xaa is A or G.
  misc_feature (5)..(5): Xaa is K or R.
  misc_feature (6)..(6): Xaa is A or G.
XXXXXX

SEQ ID NO: 7 - protein, 6 aa, Artificial Sequence
  Heparin binding motif
  MISC_FEATURE (1)..(6): Residues 1-6 can be repeated from 1 to 9 times.
AKKARA

SEQ ID NO: 8 - protein, 12 aa, Artificial Sequence
  Heparin binding motif
AKKARAAKKARA

SEQ ID NO: 9 - protein, 21 aa, Artificial Sequence
  Self-assembling monomer peptide
  MISC_FEATURE (1)..(1): Amino terminus is acylated with a C2-C30 fatty acid.
VVVVKKKKGAKKARAAKKARA

SEQ ID NO: 10 - protein, 6 aa, Artificial Sequence
  beta sheet forming segment
VVVKKK

SEQ ID NO: 11 - protein, 19 aa, Artificial Sequence
  Self-assembling monomer peptide
  MISC_FEATURE (1)..(1): Amino terminus is acylated with parlmitic acid.
VVVVKKKKGAKKARAKKAR